(19) United States
(12) Patent Application Publication
(10) Pub. No.: US 2010/0311827 A1
(43) Pub. Date: Dec. 9, 2010

(54) Chlorogenic acid derivatives and their use as anti-fungal agents

(76) Inventors: Mohsen Daneshtalab, St John's Newfoundland & Labrador (CA); Chao-Mei Ma, Beijing (CN)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/374,399

(22) PCT Filed: Jul. 23, 2007
(86) PCT No.: PCT/CA2007/001303

§ 371 Date: Jun. 9, 2009

Related U.S. Application Data
(60) Provisional application No. 60/832,409, filed on Jul. 21, 2006.

(51) Int. Cl. A61K 31/36; C07D 317/68; A01N 43/30; C07C 235/64; A61K 31/24; A01N 37/40; A01P 3/00; A61P 31/10

(52) U.S. Cl. 514/466; 549/436; 560/104; 514/534; 560/39; 514/540

(57) ABSTRACT

The invention provides chlorogenic acid derivatives of Formula (I) that are capable of inhibiting the growth of fungal cells and are useful as anti-fungal agents. The invention further provides the methods of inhibiting the growth of fungal cells and methods of treating a fungal infection in an animal by administering to the animal an effective amount of a compound of Formula I, either alone or in combination with another anti-fungal agent.

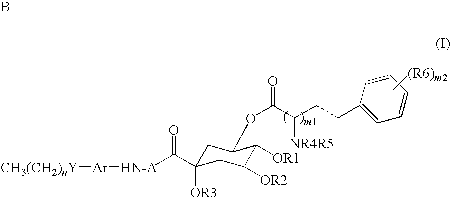

### FIELD OF THE INVENTION

The invention pertains to the field of anti-fungal compounds and, in particular, to chlorogenic acid derivatives and their use in the treatment of fungal infections.

### BACKGROUND OF THE INVENTION

Fungal infections are becoming a major health concern for a number of reasons, including the limited number of anti-fungal agents available, the increasing incidence of species resistant to older anti-fungal agents, and the growing population of immunocompromised patients at risk for opportunistic fungal infections. The most common clinical isolate is Candida albicans (comprising about 19% of all isolates). In one study, it was reported that 40% of all deaths from hospital-acquired infections were due to fungi (Sternberg, 1994, Science 266: 1632-1634).

Neutropenic patients (neutropenia due to, e.g., chemotherapy, immunosuppressive therapy, infection, including AIDS, or an otherwise dysfunctional immune system) are predisposed to the development of invasive fungal infections, most commonly including Candida species and Aspergillus species, and, on occasion, Fusarium, Trichosporon and Dreschlera. Cryptoccocus infection is also common in patients on immunosuppressive agents.

There are a large number of anti-fungal compounds or agents currently on the market that have limited clinical applications due to either the emergence of fungal resistance or unwanted adverse effects. There are three main groups of anti-fungal agents. The major group includes polyene derivatives, including amphotericin B and the structurally related compounds nystatin and pimaricin, which are only administered intravenously. These are broad-spectrum anti-fungals that bind to ergosterol, a component of fungal cell membranes, and thereby disrupt the membranes, leading to cell death. Amphotericin B is usually effective for systemic mycoses, but its administration is limited by toxic effects that include fever and kidney damage, and other accompanying side effects such as anemia, low blood pressure, headache, nausea, vomiting and phlebitis. The unrelated anti-fungal agent flucytosine (5-fluorocytosine), an orally absorbed drug, is frequently used as an adjunct to amphotericin B treatment for some forms of candidiasis and cryptococcal meningitis. Its adverse effects include bone marrow depression with leukopenia and thrombocytopenia.

The second major group of anti-fungal agents includes azole derivatives which impair synthesis of ergosterol and lead to accumulation of metabolites that disrupt the function of fungal membrane-bound enzyme systems (e.g., cytochrome P450) and inhibit fungal growth. Significant inhibition of mammalian P450 results in important drug interactions. This group of agents includes ketoconazole, clotrimazole, miconazole, econazole, butoconazole, oxiconazole, sulconazole, terconazole, fluconazole and itraconazole. These agents may be administered to treat systemic mycoses. Ketoconazole, an orally administered imidazole, is used to treat nonmeningeal blastomycosis, histoplasmosis, coccidioidomycosis and paracoccidioidomycosis in non-immunocompromised patients, and is also useful for oral and esophageal candidiasis. Adverse effects include rare drug-induced hepatitis; ketoconazole is also contraindicated in pregnancy. Itraconazole appears to have fewer side effects than ketoconazole and is used for most of the same indications. Fluconazole also has fewer side effects than ketoconazole and is used for oral and esophageal candidiasis and cryptococcal meningitis. Miconazole is a parenteral imidazole with efficacy in coccidioidomycosis and several other mycoses, but has side effects including hyperlipidemia and hyponatremia. The third major group of anti-fungal agents includes allylamines-thiocarbamates, which are generally used to treat skin infections. This group includes tolnaftate and naftifine.

Another anti-fungal agent is griseofulvin, a fungistatic agent which is administered orally for fungal infections of skin, hair or nails that do not respond to topical treatment. U.S. Pat. No. 6,355,616 describes derivatized anti-fungal compounds that are peptide-based constructs derived from or based on subsequences of Domain III (amino acids 142-169) of bactericidal/permeability-increasing protein (BPI) and in vivo and in vitro uses of such compounds. U.S. Pat. No. 6,083,921 describes a pharmaceutical composition comprising an extract or combination of extracts having an anti-viral, anti-bacterial, or immunomodulating property, wherein the extract or combination of extracts is obtained from a combination of plants. Pharmaceutical compositions comprising baicalin, chlorogenic acid and forsythiaside in isolated and purified form are also described, as are derivatives of these compounds.

Echinocandins are a further well-known group of anti-fungal compounds. Echinocandins are lipopeptides and structure-function studies have indicated that the fatty acid side chain of the Echinocandin B ring is required for anti-fungal activity. Analogues of the clinical echinocandin candidate, cilofungin, have been described (Zambias, R. A., et al., J. Med. Chem., 1992, 35:2843-2855), as have peptidomimetic analogues of echinocandins (Ma, C-H., et al., Heterocycles, 2006, 68:721-732).

This background information is provided for the purpose of making known information believed by the applicant to be of possible relevance to the invention. No admission is necessarily intended, nor should be construed, that any of the preceding information constitutes prior art against the invention.

### SUMMARY OF THE INVENTION

An object of the invention is to provide chlorogenic acid derivatives and their use as anti-fungal agents. In accordance with one aspect of the invention, there is provided a compound having the structural Formula I or a salt thereof, as an anti fungal agent:

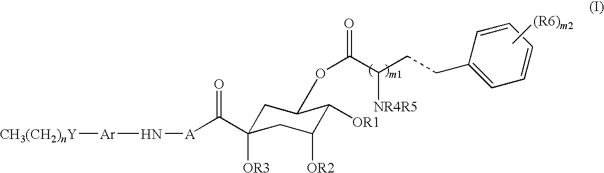

wherein:
the bond - - - is a single or a double bond;
R1, R2 and R3 are independently H, —C(O)C₁-C₆ alkyl, or C₁-C₆ alkyl; alternatively R1 and R2 are joined together to form an acetonide;
Ar is C₆-C₁₀ aryl;
Y is ONH, S, SO, or SO₂;
n is 3 to 16;
A is either absent, or 1 to 3 amino acid residues or derivatives thereof;
R4 and R5 are independently H, C₁-C₆ alkyl or an amino protecting group;
R6 is H, —OR, —OC(O)R, —C(O)OR, —NO₂ or —NR^{a}R^{b}; wherein R is H or C₁-C₆ alkyl, R^{a} and R^{b} are independently H, C₁-C₆ alkyl or an amino protecting group;

m1 is 0 or 1; and
m2 is 1 to 5.

In accordance with another aspect of the invention, there is provided a compound having the structural formula I wherein each amino acid residue or derivative thereof is:

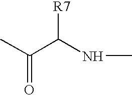

wherein R7 is —CH₂—OR, —CH(R)—OR, —CH₂—C(O)OR, —CH₂—CH₂—C(O)OR, —(CH₂)₃NR^{c}R^{d}, or —(CH₂)₄NR^{c}R^{d}, wherein R^{c} and R^{d} are independently H, C₁-C₆ alkyl, —C(O)OR or amino protecting group and R is independently H or C₁-C₆ alkyl.

In accordance with another aspect of the invention, there is provided a compound having the structural Formula II:

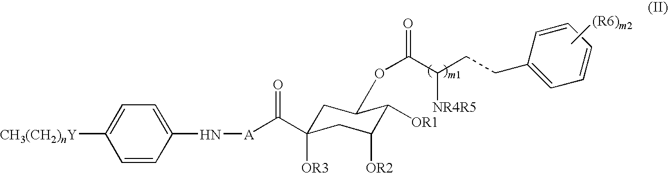

wherein the bond - - -, R1, R2, R3, R4, R5, R6, A, Y, n, m1 and m2 are as defined for Formula I.

In accordance with another aspect of the invention, there is provided a compound having the structural Formula III:

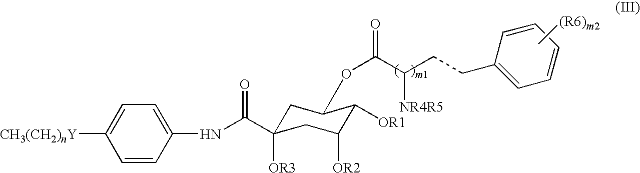

wherein the bond - - -, R1, R2, R3, R4, R5, R6, Y, n, m1 and m2 are as defined for Formula I.

In accordance with another aspect of the invention, there is provided a compound having the structural Formula IV:

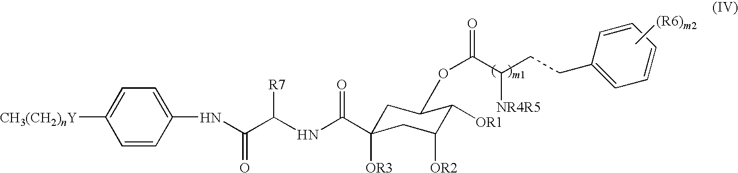

wherein the bond - - -, R1, R2, R3, R4, R5, R6, R7, Y, n, m1 and m2 are as defined for Formula I.

In accordance with another aspect of the invention, there is provided a compound having the structural Formula IVa:

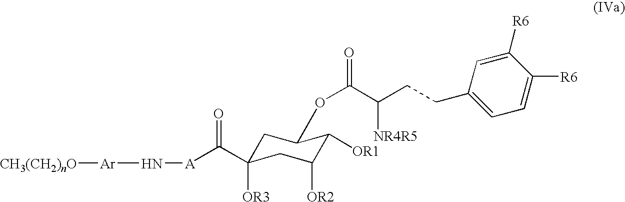

wherein the bond - - -, R1, R2, R3, R4, R5 and n are as defined for Formula I and R6 is —OR or —OC(O)R, wherein R is H or C₁-C₆ alkyl.

In accordance with another aspect of the invention there is provided a composition comprising a compound of Formula I, II, III, IV or IVa, and a carrier.

In accordance with another aspect of the invention there is provided a compound of Formula I, II, III, IV or IVa for use as an anti-fungal agent.

In accordance with another aspect of the invention there is provided a use of a compound of Formula I, II, III, IV or IVa in the preparation of a medicament.

In accordance with another aspect of the invention there is provided a method of inhibiting the growth of fungal cells comprising contacting said fungal cells with an effective amount of a compound of Formula I, II, III, IV or IVa.

In accordance with another aspect of the invention there is provided a method of treating a fungal infection in a subject, comprising administering to the subject an effective amount of the compound of Formula I, II, III, IV or IVa.

In accordance with another aspect of the invention there is provided a method of treating a fungally-related disease or disorder in a subject, comprising administering to the subject an effective amount of the compound of Formula I, II, III, IV or IVa.

In accordance with another aspect of the invention, there is provided a kit comprising a compound of Formula I, II, III, IV or IVa, and optionally instructions for use.

### BRIEF DESCRIPTION OF THE FIGURES

These and other features of the invention will become more apparent in the following detailed description in which reference is made to the appended drawings.

FIG. 1 depicts a superimposition of the structures of chlorogenic acid and echinocandin.

FIG. 2 depicts a superimposition of the structures of compound 2 and echinocandin.

### DETAILED DESCRIPTION OF THE INVENTION

The invention provides chlorogenic acid derivatives having anti-fungal activity. These chlorogenic acid derivatives are able to inhibit the growth or proliferation of fungi and thus, can be used as anti-fungal agents. The invention further provides for methods of treating a fungal infection in a subject by administering to the subject an effective amount of one or more compounds of the invention, either alone or in combination with one or more known anti-fungal agents.

Definitions

Unless defined otherwise, all technical and scientific terms used herein have the same meaning as commonly understood by one of ordinary skill in the art to which this invention belongs.

The term “alkyl” refers to a straight chain or branched alkyl group of one to six carbon atoms. This term is further exemplified by such groups as methyl, ethyl, n-propyl, i-propyl, n-butyl, t-butyl, l-butyl (or 2-methylpropyl).

The terms “aryl” or “Ar” refer to an aromatic carbocyclic group having at least one aromatic ring (e.g., phenyl or biphenyl) or multiple condensed rings in which at least one ring is aromatic.

HOBT refers to N-Hydroxybenzotriazole.

CMC refers to N-cyclohexyl-N′-(2-morpholinoethyl)carbodiimide-methyl-p-toluenesulfonate.

The term “protecting group” (such as, an amino protecting group, hydroxyl protecting group, acid protecting group and the like) refers to a functional group protecting group as known in the art, for example, as described in E. Haslam, Protecting Groups in Organic Chemistry, (J. G. W. McOmie, ed., 1973); and T. W. Greene and P. G. M. Wuts, Protective Groups in Organic Synthesis, (1991).

In the context of the present invention, the term “inhibit the growth of fungal cells” and grammatical variations thereof, means to kill or eradicate fungal cells (for example, fungicidal activity), slow or arrest the growth or proliferation of fungal cells (for example, fungistatic activity), and/or prevent fungal cell growth.

The terms “therapy” and “treatment,” as used interchangeably herein, refer to an intervention performed with the intention of alleviating the symptoms associated with, preventing the development of, or altering the pathology of a disease, disorder or condition. Thus, the terms therapy and treatment are used in the broadest sense, and include the prevention (prophylaxis), moderation, reduction, and curing of a disease, disorder or condition at various stages. Those in need of therapy/treatment include those already having the disease, disorder or condition as well as those prone to, or at risk of developing, the disease, disorder or condition and those in whom the disease, disorder or condition is to be prevented.

The term “subject” or “patient,” as used herein, refers to an animal in need of treatment.

The term “animal,” as used herein, refers to both human and non-human animals, including, but not limited to, mammals, birds and fish.

Administration of the compounds of the invention “in combination with” one or more further therapeutic agents, is intended to include simultaneous (concurrent) administration and consecutive administration. Consecutive administration is intended to encompass various orders of administration of the therapeutic agent(s) and the compound(s) of the invention to the subject.

As used herein, the term “about” refers to an approximately +/−10% variation from a given value. It is to be understood that such a variation is always included in any given value provided herein, whether or not it is specifically referred to.

I. Chlorogenic Acid Derivatives

The invention provides compounds of general Formula I:

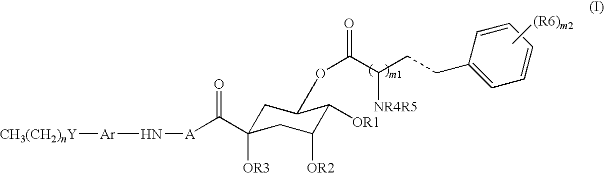

and salts thereof, wherein:

the bond - - - is a single or a double bond;

R1, R2 and R3 are independently H, —C(O)C₁-C₆ alkyl, or C₁-C₆ alkyl; alternatively R1 and R2 are joined together to form an acetonide;

Ar is C₆-C₁₀ aryl;

Y is O, NH, S, SO, or SO₂;

n is 3 to 16;

A is either absent, or 1 to 3 amino acid residues or derivatives thereof;

R4 and R5 are independently H, C₁-C₆ alkyl or an amino protecting group;

R6 is H, —OR, —OC(O)R, —C(O)OR, —NO₂ or —NR^{a}R^{b}; wherein R is H or C₁-C₆ alkyl, R^{a} and R^{b} are independently H, C₁-C₆ alkyl or an amino protecting group;

m1 is 0 or 1; and

m2 is 1 to 5.

In one embodiment of the invention, the compounds of Formula I are those wherein the 1 to 3 amino acid residues or derivatives thereof are each threonine, a derivative of threonine, ornithine, a derivative of ornithine, lysine, a derivative of lysine, serine, a derivative of serine, aspartate, a derivative of aspartate, glutamate, or a derivative of glutamate.

In another embodiment of the invention, the compounds of Formula I are those wherein each amino acid residue or derivative thereof is:

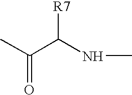

wherein R7 is —CH₂—OR, —CH(R)—OR, —CH₂—C(O)OR, —CH₂—CH₂—C(O)OR, —(CH₂)₃NR^{c}R^{d}, or —(CH₂)₄NR^{c}R^{d}, wherein R^{c} and R^{d} are independently H, C₁-C₆ alkyl, —C(O)OR or amino protecting group and R is independently H or C₁-C₆ alkyl.

In another embodiment, the compounds of Formula I are those wherein the 1 to 3 amino acid residues or derivatives thereof are each ornithine, a derivative of ornithine, lysine, or a derivative of lysine.

In another embodiment of the invention, the compounds of Formula I are those wherein R1, R2, R3, R4, R5, R6, Ar, A, Y, n, and m1 are as defined above and m2 is 1 or 2.

In another embodiment of the invention, the compounds of Formula I are those wherein m2 is 1 and the R6 group is present at the para position of the phenyl ring as shown below in Formula (Ia):

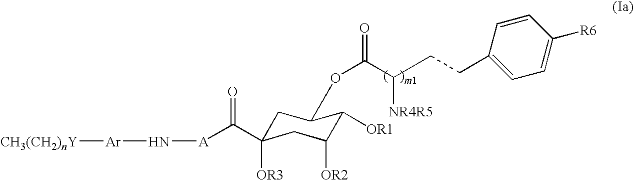

wherein the bond - - R1, R2, R3, R4, R5, R6, Ar, A, Y, n, and m1 are as defined above.

In another embodiment of the invention, the compounds of Formula I are those wherein m2 is 2 and the R6 groups are present at the 3- and 4-positions of the phenyl ring as shown below in Formula (Ib):

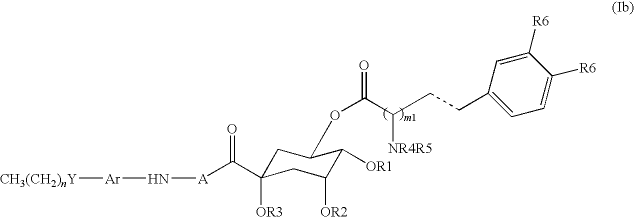

wherein the bond - - -, R1, R2, R3, R4, R5, R6, Ar, A, Y, n, and m1 are as defined above.

In another embodiment of the invention, the compounds of Formula I are those wherein the bond - - -, R1, R2, R3, R4, R5, R6, Ar, A, n, m1 and m2 are as described for Formula I and Y is O.

In another embodiment of the invention, the compound of Formula I includes compounds of structural Formula II:

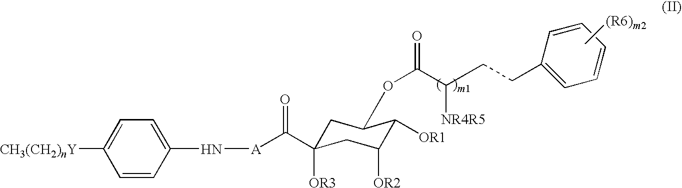

wherein the bond - - -, R1, R2, R3, R4, R5, R6, A, Y, n, m1 and m2 are as defined for Formula I.

In another embodiment of the invention, the compound of Formula II includes compounds of structural Formula III:

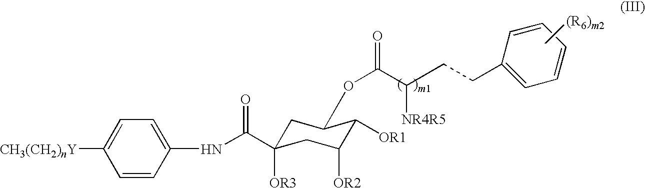

wherein the bond - - -, R1, R2, R3, R4, R5, R6, Y, n, m1 and m2 are as defined for Formula I.

In another embodiment of the invention, the compound of Formula II includes compounds of structural Formula IV:

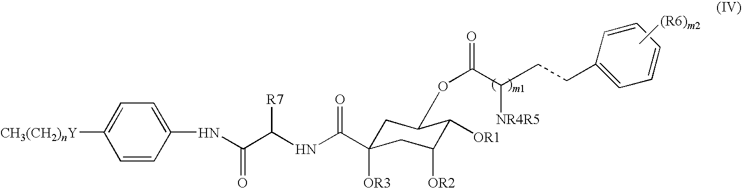

wherein the bond - - -, R1, R2, R3, R4, R5, R6, R7, Y, n, m1 and m2 are as defined for Formula I.

In another embodiment of the invention, the compounds of Formulae II, III and IV are those wherein the bond - - -, R1, R2, R3, R4, R5, R6, R7 (when present), A (when present), Y, n and m1 are as defined above and m2 is 1 or 2.

In another embodiment of the invention, the compounds of Formulae II, III and IV are those wherein the bond - - -, R1, R2, R3, R4, R5, R5, R6, R7 (when present), A (when present), Y, n and m1 are as defined above, and m2 is 1 and the R6 group is present at the para position of the phenyl ring.

In another embodiment of the invention, the compounds of Formulae II, III and IV are those wherein the bond - - -, R1, R2, R3, R4, R5, R5, R6, R7 (when present), A (when present), Y, n and m1 are as defined above, and m2 is 2 and the R6 groups are present at the 3- and 4-positions of the phenyl ring.

In another embodiment of the invention, the compounds of Formulae II, III and IV are those wherein the bond - - -, R1, R2, R3, R4, R5, R5, R6, R7 (when present), A (when present), n, m 1 and m2 are as defined above, and Y is O.

In another embodiment of the invention, the compounds of Formulae I, II, III and IV are those wherein the bond - - -, R3, R4, R5, R5, R6, R7 (when present), A (when present), n, m1 and m2 are as defined above, Y is O and R1 and R2 are each H.

In another embodiment of the invention, the compounds of Formulae I, II, III and IV are those wherein Y is O, R1, R2, R3, R4, R5 are each H and R6 is OH.

In another embodiment of the invention, the compound of Formula I includes the compound of structural Formula IVa:

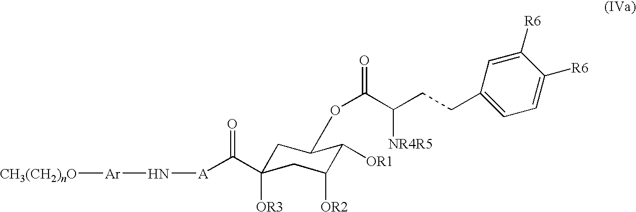

wherein the bond - - -, R1, R2, R3, R4, R5 and n are as defined for Formula I, and R6 is —OR or —OC(O)R, wherein R is H or C₁-C₆ alkyl.

In another embodiment of the invention, in the compound of Formula IVa, R6 is OR, wherein R is H or C₁-C₆ alkyl.

In another embodiment of the invention, in the compound of Formula IVa, R6 is OH.

In another embodiment, compounds of the invention include, but are not limited to, the following exemplary compounds:

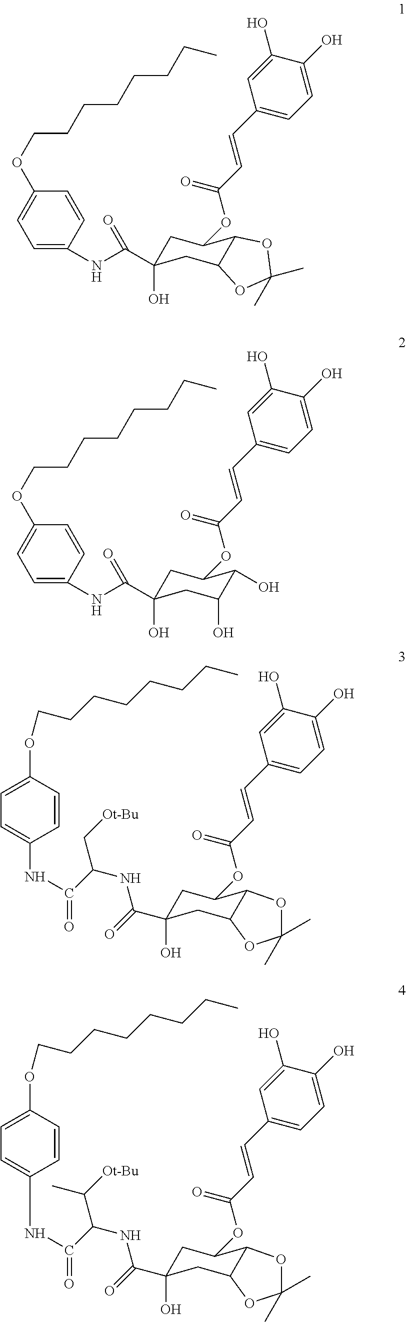

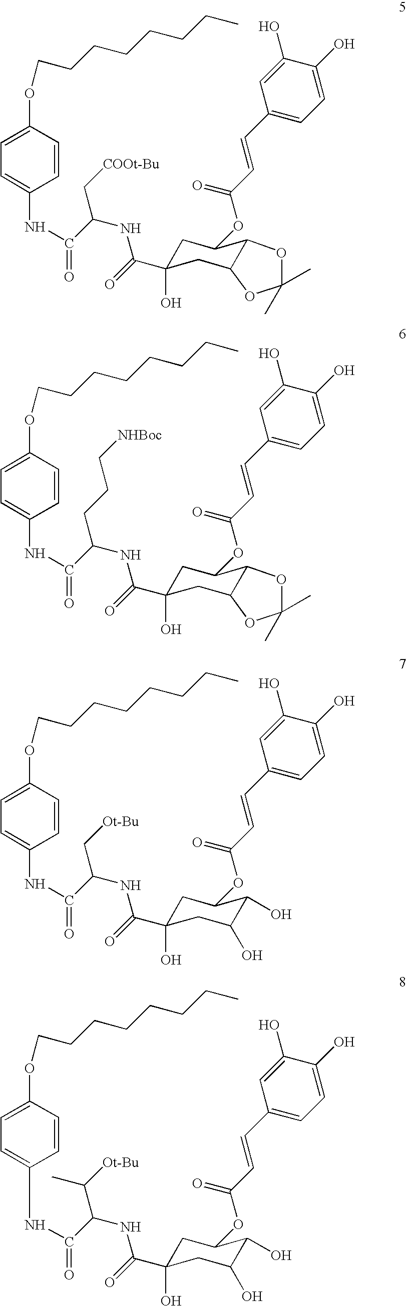

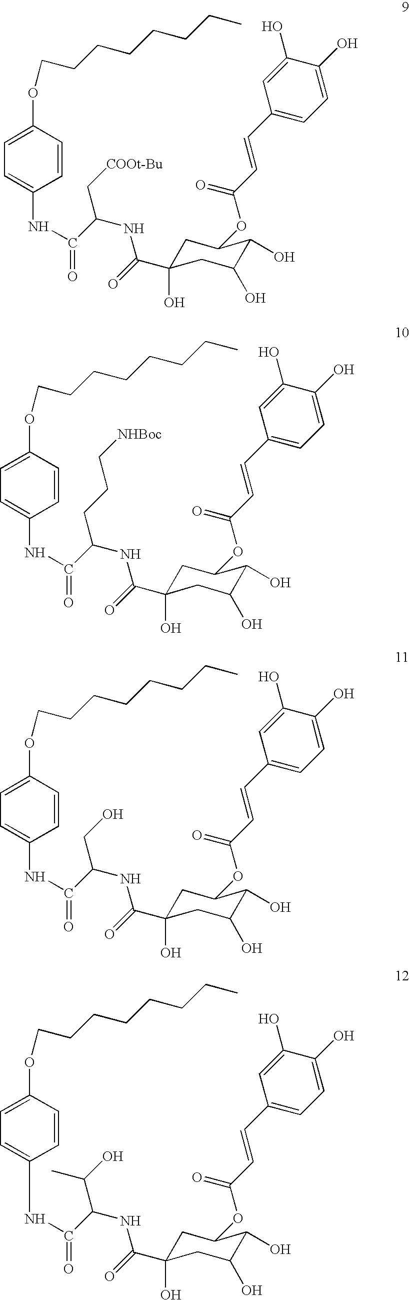

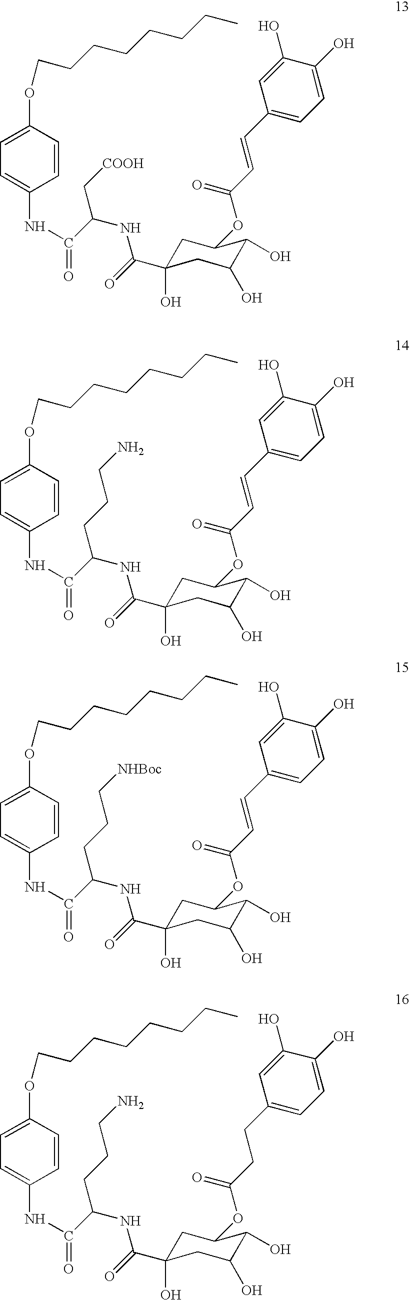

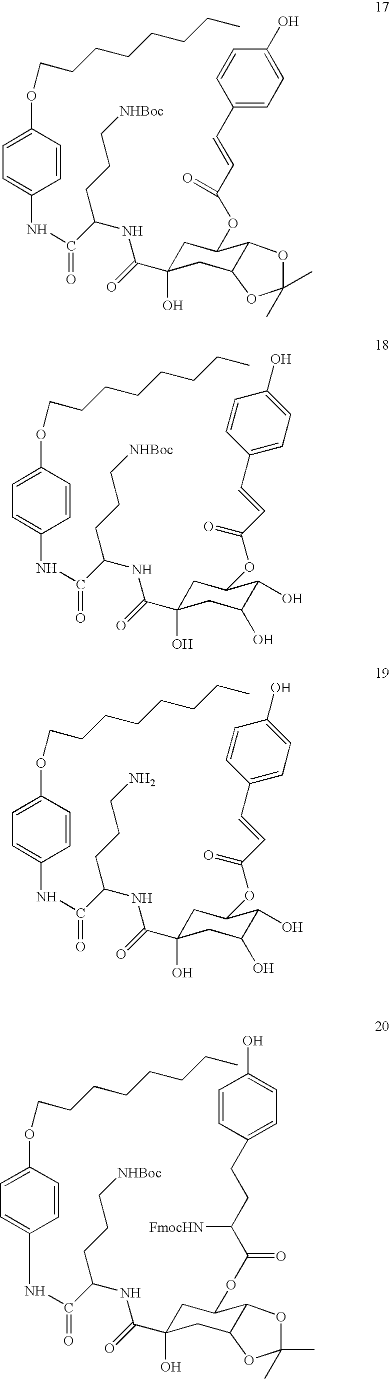

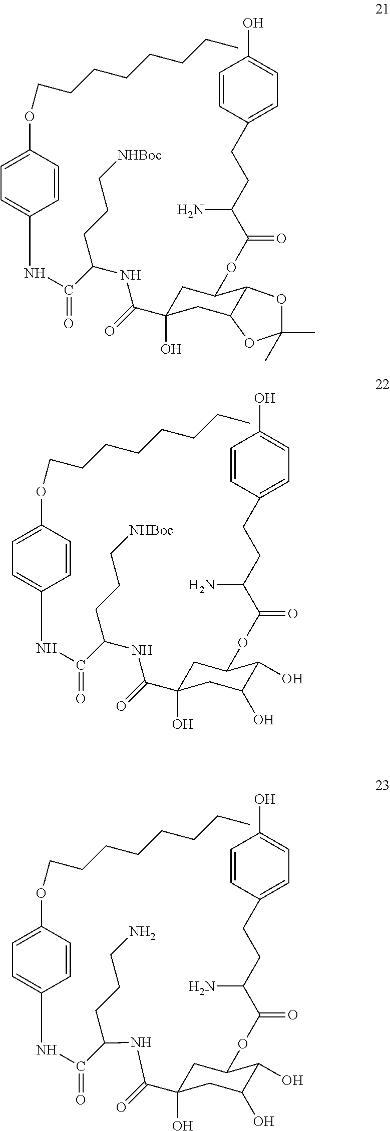

The invention includes pharmaceutically acceptable salts of the compounds defined by Formula I. Compounds according to the invention can possess a sufficiently acidic group, a sufficiently basic group, or both functional groups, and accordingly react with a number of organic and inorganic bases, and organic and inorganic acids, to form pharmaceutically acceptable salts.

The term “pharmaceutically acceptable salt” as used herein, refers to a salt of a compound of Formula I, which is substantially non-toxic to living organisms. Typical pharmaceutically acceptable salts include those salts prepared by reaction of the compound of the invention with a pharmaceutically acceptable mineral or organic acid or an organic or inorganic base. Such salts are known as acid addition and base addition salts.

Acids commonly employed to form acid addition salts are inorganic acids such as hydrochloric acid, hydrobromic acid, hydroiodic acid, sulphuric acid, phosphoric acid, and the like, and organic acids such as p-toluenesulphonic acid, methanesulphonic acid, oxalic acid, p-bromophenylsulphonic acid, carbonic acid, succinic acid, citric acid, benzoic acid, acetic acid, and the like. Examples of such pharmaceutically acceptable salts are the sulphate, pyrosulphate, bisulphate, sulphite, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, hydroxybenzoate, methoxybenzoate, phthalate, xylenesulphonate, phenylacetate, phenylpropionate, phenylbutyrate, citrate, lactate, gamma-hydroxybutyrate, glycolate, tartrate, methanesulphonate, propanesulphonate, naphthalene-1-sulfonate, napththalene-2-sulfonate, mandelate and the like. Preferred pharmaceutically acceptable acid addition salts are those formed with mineral acids such as hydrochloric acid and hydrobromic acid, and those formed with organic acids such as maleic acid and methanesulphonic acid.

Salts of amine groups may also comprise quarternary ammonium salts in which the amino nitrogen carries a suitable organic group such as an alkyl, lower alkenyl, substituted lower alkenyl, lower alkynyl, substituted lower alkynyl, or aralkyl moiety.

Base addition salts include those derived from inorganic bases, such as ammonium or alkali or alkaline earth metal hydroxides, carbonates, bicarbonates, and the like. Bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like.

One skilled in the art will understand that the particular counterion forming a part of a salt of this invention is usually not of a critical nature, so long as the salt as a whole is pharmacologically acceptable and as long as the counterion does not contribute undesired qualities to the salt as a whole. The invention further encompasses the pharmaceutically acceptable solvates of a compound of Formula I. Many of the compounds of Formula I can combine with solvents such as water, methanol, ethanol and acetonitrile to form pharmaceutically acceptable solvates such as the corresponding hydrate, methanolate, ethanolate and acetonitrilate.

The compounds of the invention may have multiple asymmetric (chiral) centres. As a consequence of these chiral centres, the compounds of the invention occur as racemates, mixtures of enantiomers and as individual enantiomers, as well as diastereomers and mixtures of diastereomers. All asymmetric forms, individual isomers and combinations thereof, are within the scope of the invention.

Non-toxic metabolically-labile esters or amides of a compound of Formula I are those that are hydrolysed in vivo to afford the compound of Formula I and a pharmaceutically acceptable alcohol or amine. Examples of metabolically-labile esters include esters formed with (1-6C) alkanols, in which the alkanol moiety may be optionally substituted by a (1-8C) alkoxy group, for example methanol, ethanol, propanol and methoxyethanol. Non-limiting examples of metabolically-labile amides include amides formed with amines such as methylamine.

II. Preparation of Compounds of Formula I

According to another aspect, the invention provides a process for the preparation of a compound of Formula I.

In one embodiment of the invention, compounds of Formula I, wherein R1 and R2 are H, can be prepared by hydrolyzing a compound of Formula I, wherein R1 and R2 are joined together to form an acetonide, under acidic conditions in the presence of a suitable solvent, wherein R3, R4, R5, R6, Ar, A, Y, n, m1, m2 and the bond - - - are as defined above.

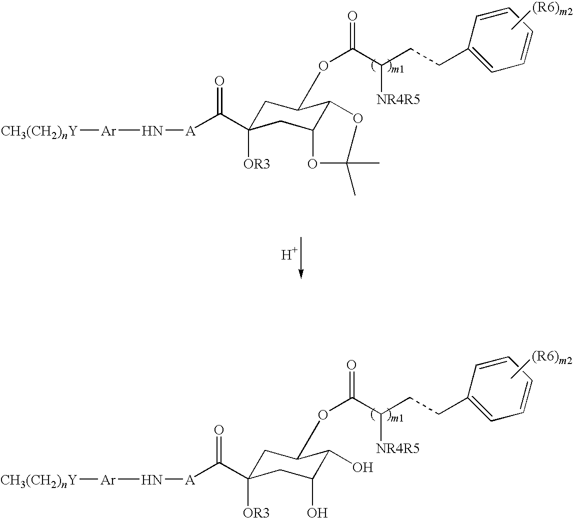

In another embodiment, compounds of Formula I wherein R1 and R2 are —C(O)C₁-C₆ alkyl or C₁-C₆ alkyl, can be prepared by reacting a compound of Formula I wherein R1 and R2 are H, with an appropriate acylating or alkylating agent, under suitable conditions within the knowledge of a worker skilled in the art, wherein R3, R4, R5, R6, Ar, A, Y, n, m1, m2 and bond - - - are as defined above or R3 is an hydroxy protecting group or one of R4 and R5 is an amino protecting group.

In another embodiment, compounds of Formula I wherein the bond - - - is a single bond, can be prepared by hydrogenating a compound of Formula I, wherein the bond - - - is a double bond, under suitable conditions, for example, in the presence of H₂/Pd—C, wherein R1, R2 R3, R4, R5, R6, Ar, A, Y, n, m1 and m2 are as defined above.

In another embodiment, compounds of Formula I, wherein A is absent, R1 and R2 are joined together to form an acetonide and R3, R4, R5, R6, Ar, Y, n, m1, m2 and the bond - - - are as defined above, can be prepared by reacting a compound of Formula V, wherein R3, R4, R5, R6, m1, m2 and the bond - - - are as defined above, with CH₃(CH₂)ₙY—Ar—NH₂, wherein Ar, Y, and n are as defined above, in the presence of suitable coupling agent(s) and solvent(s):

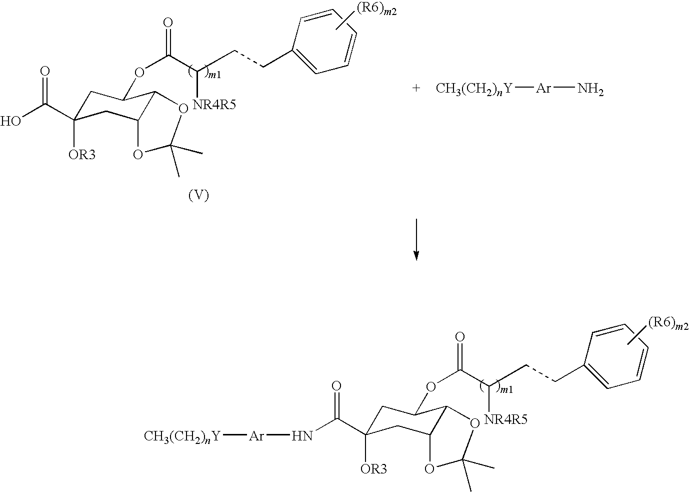

In another embodiment, compounds of Formula I wherein R1 and R2 are H, A is an amino acid derivative as defined above where R7 is —CH₂—OH, —CH(R)—OH, —CH₂—C(O)OH, —CH₂—CH₂—C(O)OH, —(CH₂)₃NH₂, or —(CH₂)₄NH₂, can be prepared by deprotecting a compound of Formula I wherein A is an amino acid derivative as defined above where R7 is —CH₂—OR, —CH(R)—OR, —CH₂—C(O)OR, —CH₂—CH₂—C(O)OR, —(CH₂)₃NR^{c}R^{d}, or —(CH₂)₄NR^{c}R^{d}, where R is C₁-C₆ alkyl, R^{c} and R^{d} are independently H, C₁-C₆ alkyl, —C(O)C₁-C₆ alkyl or an amino protecting group, wherein when R^{c} is H, then R^{d} is C₁-C₆ alkyl, —C(O)C₁-C₆ alkyl or an amino protecting group or vice versa, wherein R3, R4, R5, R6, Ar, Y, n, m1, m2, and the bond - - - are as defined above.

Compounds of Formula I wherein R1 and R2 are H, A is an amino acid derivative as defined above where R7 is —CH₂—OR, —CH(R)—OR, —CH₂—C(O)OR, —CH₂—CH₂—C(O)OR, —(CH₂)₃NR^{c}R^{d}, or —(CH₂)₄NR^{c}R^{d}, where R is C₁-C₆ alkyl, R^{c} and R^{d} are independently H, C₁-C₆ alkyl, —C(O)C₁-C₆ alkyl or an amino protecting group, wherein when R^{c} is H, R^{d} is C₁-C₆ alkyl, —C(O)C₁-C₆ alkyl or an amino protecting group or vice versa, can be prepared by hydrolyzing, under suitable conditions, a compound of Formula I, wherein R1 and R2 are joined together to form an acetonide and A is an amino acid derivative wherein R7 is —CH₂—OR, —CH(R)—OR, —CH₂—C(O)OR, —CH₂—CH₂—C(O)OR, —(CH₂)₃NR^{c}R^{d}, or —(CH₂)₄NR^{c}R^{d} and where R is C₁-C₆ alkyl, R^{c} and R^{d} are independently H, C₁-C₆ alkyl, —C(O)C₁-C₆ alkyl or an amino protecting group, wherein when R^{c} is H, then R^{d} is C₁-C₆ alkyl, —C(O)C₁-C₆ alkyl or an amino protecting group or vice versa, wherein R3, R4, R5, R6, Ar, Y, n, m1, m2 and the bond - - - are as defined above.

Compounds of Formula I wherein R1 and R2 are joined together to form an acetonide and A is an amino acid derivative wherein R7 is —CH₂—OR, —CH(R)—OR, —CH₂—C(O)OR, —CH₂—CH₂—C(O)OR, —(CH₂)₃NR^{c}R^{d}, or —(CH₂)₄NR^{c}R^{d} and where R is C₁-C₆ alkyl, R^{c} and R^{d} are independently H, C₁-C₆ alkyl, —C(O)C₁-C₆ alkyl or an amino protecting group, wherein when R^{c} is H, then R^{d} is C₁-C₆ alkyl, —C(O)C₁-C₆ alkyl or an amino protecting group or vice versa, can be obtained by reacting a compound of Formula V, wherein R3, R4, R5, R6, m1, m2 and the bond - - - are defined as above, with a compound of Formula VI, wherein R7 is —CH₂—OR, —CH(R)—OR, —CH₂—C(O)OR, —CH₂—CH₂—C(O)OR, —(CH₂)₃NR^{c}R^{d}, or —(CH₂)₄NR^{c}R^{d}, where R is C₁-C₆ alkyl, R^{c} and R^{d} are independently H, C₁-C₆ alkyl, —C(O)C₁-C₆ alkyl or an amino protecting group, wherein when R^{c} is H, then R^{d} is C₁-C₆ alkyl, —C(O)C₁-C₆ alkyl or an amino protecting group or vice versa, wherein R3, R4, R5, R6, Ar, Y, n, m1, m2 and the bond - - - are as defined above, in the presence of suitable coupling agent(s) and solvents as follows:

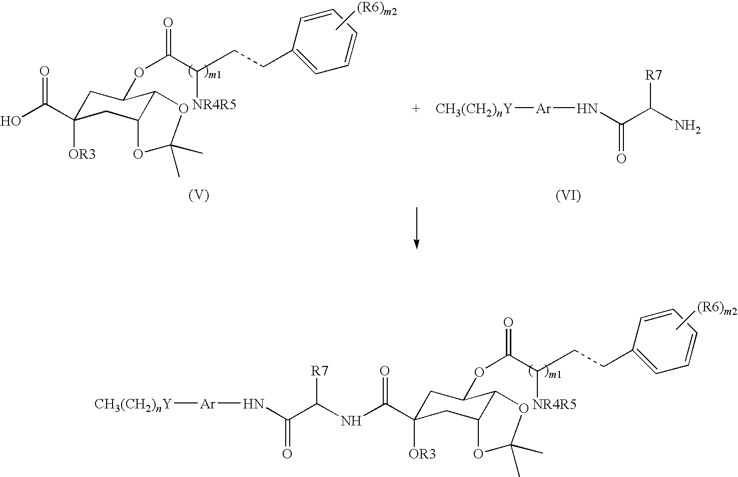

Compounds of Formula V can be prepared by reacting a compound of Formula VII with dry acetone in the presence of conc. H₂SO₄ as follows, wherein R3, R4, R5, R6, m1, m2 and the bond - - - are as defined above:

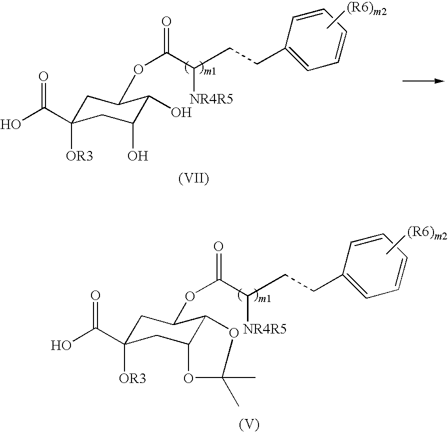

Compounds of Formula VI can be prepared by reacting a compound of Formula (VIII), wherein R7 is as defined above and R8 is an amino protecting group, with CH₃(CH₂)ₙY—Ar—NH₂, followed by deprotecting the amino group NHR8, wherein Ar, Y, and n are as defined above:

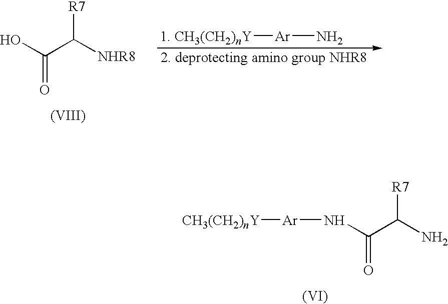

Compounds of Formula VII can be prepared from a compound of Formula IX:
a) The compounds of Formula VII (for example compounds of Formula VIIa), wherein m1 is absent, the bond - - - is a double bond, R3 is H, R6 and m2 are as defined above, can be obtained by reacting a compound of Formula IX with a compound of Formula X, followed by hydrolyzing the reaction mixture under acidic conditions, wherein R6 and m2 are as defined above.

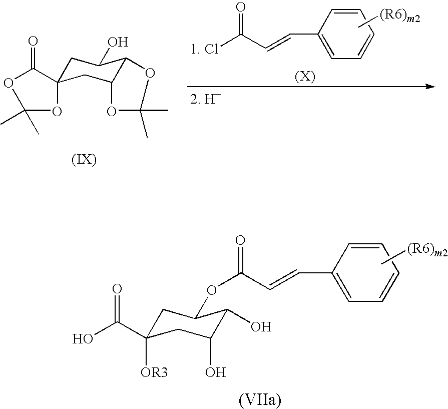

b) The compounds of Formula VII (for example compounds of Formula VIIb), wherein m1 is present, the bond - - - is single bond, R3 is H, R6 and m2 are as defined above and R4 is H, R5 is C₁-C₆ alkyl or an amino protecting group or vice versa, can be obtained by reacting a compound of Formula IX with a compound of Formula XI followed by hydrolyzing the reaction mixture under acidic conditions, wherein R4, R5, R6, and m2 are as defined above.

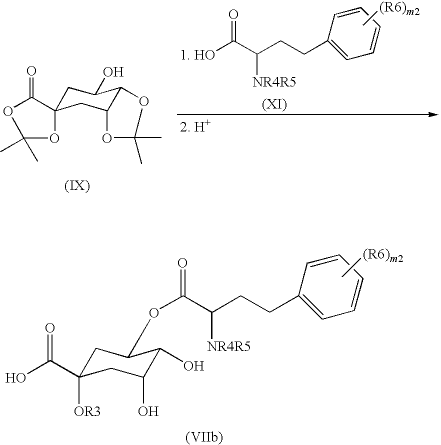

Compound of Formula IX can be prepared from the compound of Formula XIII via the following procedure as described by Sefkow, M. in Eur. J. Org. Chem, 2001:1137:

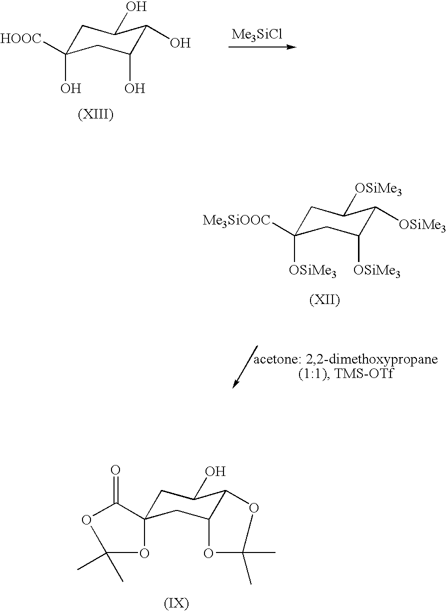

III. Anti-fungal Activity of Compounds of Formula I

The anti-fungal activity of a candidate compound of Formula I can be tested using standard techniques known in the art to determine whether the compound is suitable as a compound of the invention. As is known in the art, anti-fungal activity of a compound may result in the killing or eradication of fungal cells (i.e. fungicidal activity), in the slowing or arrest of the growth or proliferation of fungal cells (i.e. fungistatic activity), and/or in the prevention of fungal cell growth. Thus, the compounds of the invention may be fungicidal, fungistatic, and/or may prevent the growth of fungal cells. Compounds of the invention that slow or arrest fungal cell growth may be useful in combination treatments with other known anti-fungal agents. Exemplary methods of testing candidate compounds of Formula I are provided below and in the Examples included herein. These methods can be used to test the anti-fungal activity of candidate compounds of Formula I alone, or in combination with other anti-fungal agents. One skilled in the art will understand that other methods of testing the anti-fungal activity of compounds are known in the art and are also suitable for testing candidate compounds.

A. In Vitro Testing

In vitro methods of determining the ability of candidate compounds to inhibit the growth of fungal cells are well-known in the art. In general, these methods involve contacting a culture of the cells of interest with various concentrations of the candidate compound and monitoring the growth of the cell culture relative to an untreated control culture. A second control culture comprising cells contacted with a known anti-fungal agent may also be included in such tests, if desired.

For example, the ability of a candidate compound of Formula I to inhibit the growth of fungal cells can readily be determined by measurement of the minimum inhibitory concentration (MIC) for the compound. The MIC is defined as the lowest concentration that inhibits growth of the organism to a pre-determined extent. For example, a MIC₁₀₀ value is defined as the lowest concentration that completely inhibits growth of the organism, whereas a MIC₉₀ value is defined as the lowest concentration that inhibits growth by 90% and a MIC₅₀ value is defined as the lowest concentration that inhibits growth by 50%. MIC values are sometimes expressed as ranges, for example, the MIC₁₀₀ for a compound may be expressed as the concentration at which no growth is observed or as a range between the concentration at which no growth is observed and the concentration of the dilution which immediately follows.

Techniques for determining anti-fungal MIC values for candidate compounds include both macrodilution and microdilution methods (see, for example, Pfaller, M. A., Rex, J. H., Rinaldi, M. G., Clin. Infect. Dis., (1997) 24:776-84). As is known in the art, different types of fungi may require different testing methods. For example, suitable reference methods for testing MIC values for candidate compounds in yeasts, include the NCCLS reference method for broth dilution anti-fungal susceptibility testing of yeasts (approved standard-second edition, M27-A2, National Committee for Clinical Laboratory Standards, Villanova, Pa. 2003). This method can be used to test MIC values in yeasts such as Candida species, and Cryptococcus neoformans, for example. Alternatively, suitable reference methods for determining MIC values for candidate compounds in filamentous fungi include the NCCLS reference method for broth dilution anti-fungal susceptibility testing of filamentous fungi (approved standard, M38-A, National Committee for Clinical Laboratory Standards, Villanova, Pa. 2002). The latter method is suitable for determining MIC values in species of yeast such as Aspergillus, Fusarium, Rhizopus, Pseudallescheria boydii, and the mycelial from of Sporothrix schenckii.

In the classical broth microdilution method, the candidate anti-fungal compound is diluted in culture medium in a sterile, covered 96-well microtiter plate. An overnight culture of a single fungal colony is diluted in sterile medium such that, after inoculation, each well in the microtiter plate contains an appropriate number of colony forming units (CFU)/ml (typically, approximately 5×10⁵ CFU/ml). Culture medium only (containing no fungal cells) is also included as a negative control for each plate and known anti-fungal compounds are often included as positive controls. The inoculated microtiter plate is subsequently incubated at an appropriate temperature (for example, 35° C.-37° C. for 16-48 hours). The turbidity of each well is then determined by visual inspection and/or by measuring the absorbance, or optical density (OD), at 595 nm or 600 nm using a microplate reader and is used as an indication of the extent of fungal growth.

In accordance with one embodiment of the invention, a compound of Formula I is considered to have an anti-fungal effect against a given fungus when the MIC of the compound (when used alone) for 80% inhibition of growth of the fungus is about 75 μg/ml or less. In one embodiment, a compound of Formula I is considered to have an anti-fungal effect against a given fungus when the compound has a MIC for 80% inhibition of growth of about 64 μg/ml or less. In another embodiment, a compound of Formula I is considered to have an anti-fungal effect against a given fungus when the compound has a MIC for 80% inhibition of growth of about 50 μg/ml or less. In another embodiment, a compound of Formula I is considered to have an anti-fungal effect against a given fungus when the compound has a MIC for 80% inhibition of growth of about 35 μg/ml or less. In other embodiments, a compound of Formula I is considered to have an anti-fungal effect against a given fungus when the compound has a MIC for 80% inhibition of growth of about 25 μg/ml or less, about 16 μg/ml or less and about 12.5 μg/ml or less.

Anti-fungal effects may also be expressed as the percentage (%) inhibition of growth of a given fungus over a pre-determined period of time by treatment with a single concentration of a candidate compound. This method provides a rapid method of assessing the ability of a compound to inhibit fungal growth, for example, prior to conducting more in-depth tests, such as MIC determinations or in vivo testing. The predetermined period of time depends on the given fungus being tested. Thus in one embodiment, the ability of a candidate compound to inhibit fungal cell growth is tested over a predetermined amount of time of between about 18 to about 24 hours. In another embodiment, the ability of a candidate compound to inhibit fungal cell growth is tested over a predetermined amount of time of about 48 hours. In yet another embodiment, the ability of a candidate compound to inhibit fungal cell growth is tested over a predetermined amount of time of between about 48 to about 72 hours.

In one embodiment of the invention, a candidate compound is considered to be a potential anti-fungal agent when it is capable of inhibiting the growth of a given fungus by about 25% when used at a concentration of about 25 μg/ml, with growth of the fungus being assessed over the appropriate predetermined amount of time. In another embodiment, a candidate compound is considered to be a potential anti-fungal agent when it is capable of inhibiting the growth of a given fungus by about 50% when used at a concentration of about 25 μg/ml, with growth of the fungus being assessed over the appropriate predetermined amount of time. In another embodiment, a candidate compound is considered to be a potential anti-fungal agent when it is capable of inhibiting the growth of a given fungus by about 75% when used at a concentration of about 25 μg/ml, with growth of the fungus being assessed over the appropriate predetermined amount of time. In another embodiment, a candidate compound is considered to be a potential anti-fungal agent when it is capable of inhibiting the growth of a given fungus by about 80% when used at a concentration of about 25 μg/ml, with growth of the fungus being assessed over the appropriate predetermined amount of time.

One skilled in the art will appreciate that compounds that exhibit poor anti-fungal activity when used alone may still be capable of good anti-fungal activity when used in combination with one or more known anti-fungal agents. For example, the compound may sensitise the fungal cells to the action of the other agent(s), it may act in synergy with agent(s), or it may otherwise potentiate the activity of the agent(s). Compounds of the invention thus include compounds that exhibit poor activity as sole agents but good activity in combination with other anti-fungal agents. The ability of a candidate compound to exert an effect in combination with a known anti-fungal agent can be tested using standard methods, such as those described above. In addition, the ability of a compound of the invention to exhibit a synergistic effect in combination with another anti-fungal agent can be tested by standard methods, such as the measurement of the fractional inhibitory concentration (FIC) index.

B. In Vivo Testing

The ability of a candidate compound of Formula I to act as an anti-fungal agent can also be tested in vivo using standard techniques. A number of animal models are known in the art that are suitable for testing the activity of anti-fungal compounds and are readily available.

Representative examples of animal models suitable for testing the anti-fungal activity of a compound of Formula I in vivo include, but are not limited to, the severe combined immunodeficiency (SCID) mouse model and a colostrum-deprived SPF piglet model for Cryptosporidium parvum infection, a granulocytopenic rabbit model of disseminated Candidiasis (see, for example, Walsh, et al., J. Infect. Dis., 1990, 161:755-760; Thaler, et al., J. Infect. Dis., 1988, 158:80), a mouse model of disseminated Aspergillosis (see, for example, Arroyo, et al., Antimicrob. Agents Chemoth., 1977, pp. 21-25) and a neutropenic rat model of disseminated Candidiasis (see, for example, Lechner, et al., Am. J. Physiol. (Lung Cell. Mol. Physiol.) 1994, 10:1-8). The compounds of Formula I can also be tested in vivo using a comparative survival efficacy study in mice systemically infected with C. albicans (as described in U.S. Pat. No. 6,335,616).

Methods for conducting in vivo tests to determine the activity of anti-fungal compounds are well-known in the art. Typically, in vivo testing comprises introducing a selected fungus into the appropriate animal model in a sufficient amount to cause infection, followed by administration of one or more doses of the candidate compound of Formula I. Methods of administration will vary depending on the compound being employed, but can be, for example, by way of bolus infusion into a suitable vein (such as the tail vein of mice or rats), or by oral administration. Animals treated with a known anti-fungal agent and/or with a saline or buffer control solution serve as controls. Repeat doses of the test compound may be administered to the animal, if necessary, at appropriate time intervals. The animals are subsequently monitored daily for mortality.

When tested by such methods, a compound of Formula I is considered to exert an in vivo anti-fungal effect if it results in a decrease in mortality of at least about 15% in treated animals compared to test animals. In one embodiment of the invention, a compound of Formula I is considered to exert an in vivo anti-fungal effect if it results in a decrease in mortality of at least about 25% in the treated animals. In another embodiment, a compound of Formula I is considered to exert an in vivo anti-fungal effect if it results in a decrease in mortality of at least about 40% in the treated animals. In other embodiments, a compound of Formula I is considered to exert an in vivo anti-fungal effect if it results in a decrease in mortality of at least about 50%, at least about 60%, at least about 70%, at least about 80% and at least about 90% in the treated animals.

IV. Toxicity Testing

In some contexts, for example when used in vivo, it is important that the anti-fungal compounds of the invention exhibit low toxicity. As such, the compounds of Formula I may be submitted to toxicity tests, if desired, to determine their suitability for in vivo use. Toxicity tests for potential drugs are well-known in the art (see, for example, Hayes, A. W., ed., (1994), Principles and Methods of Toxicology, 3^{rd} ed., Raven Press, NY; Maines, M., ed., Current Protocols in Toxicology, John Wiley & Sons, Inc., NY).

The general in vitro toxicity of these compounds can be tested using the Brine Shrimp Lethality assay, as described in Mclaughlin, J. L. in Methods in Plant Biochemistry. Ed. K. Hostettmann, Academic Press, London, 1991; Vol. 6, pp. 1-32. Typically, candidate compounds are diluted in seawater to a suitable concentration and added to second instar larvae of Artemia salina. After a suitable period of time, for example 24 hours, the survivors are counted. In vitro acute toxicity testing of a compound of Formula I can also be performed using mammalian cell lines (see, for example, Ekwall, B., Ann. N.Y. Acad. Sci., (1983) 407:64-77). Selection of an appropriate cell line is dependent on the potential application of the candidate compound and can be readily determined by one skilled in the art.

In vivo toxicity testing can be performed by standard methodology, for example, by injecting varying concentrations of the candidate compound into an appropriate animal model. The compound can be injected once, or administration can be repeated over several days. The toxic effects of the compound can be evaluated over an appropriate time period by monitoring the general health and body weight of the animals. After the completion of the period of assessment, the animals can be sacrificed and the appearance and weight of the relevant organs determined.

In accordance with one embodiment of the invention, a compound of Formula I for use in vivo shows both good anti-fungal activity, alone or in combination with another anti-fungal agent, and low or no toxicity at the concentration at which it would be administered as an anti-fungal agent.

V. Uses of the Anti-Fungal Compounds of Formula I

The invention provides for the use of one or more compounds of the invention for the inhibition, prevention or eradication of the growth and/or proliferation of fungi, either alone or in combination with one or more known anti-fungal agents.

In one embodiment, the invention provides a method of inhibiting fungal growth by contacting a fungus with an effective amount of one or more compounds of the invention either alone or in combination with one or more other anti-fungal agents. Representative examples of fungi that may be inhibited with compounds of the invention include, but are not limited to, Histoplasma (e.g. H. capsulstum), Coccidioides, Blastomyces, Paracoccidioides, Cryptococcus (e.g. C. neoformans), Aspergillus (e.g. A. fumigatus, A. flaws, A. niger, A. nidulans, A. terreus, A. sydowi, A. flavatus, and A. glaucus), Zygomycetes (e.g. Basidiobolus, Conidiobolus, Rhizopus, Mucor, Absidia, Mortierella, Cunninghamella, and Saksenaea), Candida (e.g. C. albicans, C. tropicalis, C. parapsilosis, C. stellatoidea, C. krusei, C. parakrusei, C lusitaniae, C. pseudorropicalis, C. guilliermondi and C. glabrata), Cryptosporidium parvum, Sporothrix schenckii, Piedraia hortae, Trichosporon beigelii, Malassezia furfur, Phialophora verrucosa, Fonsecae pedrosoi, Madurella mycetomatis and Pneumocystis carinii.

In one embodiment, the compounds according to the invention have anti-fungal activity against yeasts. Examples of yeasts that are susceptible to the anti-fungal effects of these compounds include, but are not limited to Candida sp. and Cryptococcus sp. In one embodiment, the compounds according to the invention exhibit anti-fungal activity including activity against Cryptococcus neoformans. In another embodiment, the compounds according to the invention exhibit anti-fungal activity including activity against Candida albicans. In still another embodiment, the compounds according to the invention exhibit anti-fungal activity including activity against Candida krusei.

In another embodiment, the compounds according to the invention have anti-fungal activity that includes activity against filamentous fungi. Examples of filamentous fungi include, but are not limited to Aspergillus sp., Fusarium sp., and Rhizopus sp. In one embodiment, the compounds according to the invention exhibit anti-fungal activity including activity against Aspergillus sp. In another embodiment, the compounds according to the invention exhibit anti-fungal activity including activity against Aspergillus fumigatus.

In another embodiment, the compounds of the invention exhibit anti-fungal activity against a broad spectrum of fungi and are suitable for use as broad-spectrum anti-fungal agents. One embodiment of the invention thus provides for the use of compounds of Formula I as broad spectrum anti-fungal agents. Another embodiment of the invention provides for the use of compounds of Formula IVa as broad spectrum anti-fungal agents.

In accordance with another embodiment, the compounds according to the invention are capable of inhibiting the growth of or killing dermatophytes. Examples of dermatophytes include, but are not limited to, Epidermophyton sp., Microsporum sp., and Trichophyton sp.

In accordance with one embodiment of the invention, one or more compounds of the invention can be administered in a therapeutically effective amount either alone or in combination with other anti-fungal agents to a subject with a fungal infection. Such subjects may be suffering from invasive and deep-seated fungal infections or are subjects that are at high risk of contracting such an infection. Fungal infections are common, for example, in patients with HIV/AIDS and in cancer patients undergoing chemotherapy. In one embodiment, the compounds of the invention can be used in the treatment of fungal infections in such patients.

In accordance with another embodiment of the invention, one or more compounds of the invention can be used alone or in combination with other anti-fungal agents to treat a subject having a fungally-related disorder or disease. Examples of fungally-related disorders and diseases include, but are not limited to, Candidiasis; endemic mycoses (such as Histoplasmosis, Coccidioidomycosis, Blastomycosis, Paracoccidioidomycosis, Cryptococcosis, Aspergillosis, Mucormycosis), associated disseminated infections and progressive pulmonary disease; cryptococcal meningitis; narcotising patchy bronchopneumonia; haemorrhagic pulmonary infarction; rhinocerebral disease; neutropenia, black piedra; white piedra; tinea (versicolor, capitis, corporis, and the like); Pneumocystis pneumonia; chromoblastomycosis, and maduromycosis.

One embodiment of the invention provides for the use of the anti-fungal compounds of the invention in the treatment of subjects having an infection with a species of Candida., Cryptococcus, or Aspergillus, or having a disease or disorder related to infection with a species of Candida, Cryptococcus, or Aspergillus, Examples of diseases and disorders related to Candida infection include, but are not limited to, candidiasis, monilia, thrush, skin rash, diaper rash, nailbed infections and esophagitis. Examples of diseases and disorders related to Cryptococcus infection include, but are not limited to, cryptococcosis, meningitis, hepatitis, osteomyelitis, prostatitis, pyelonephritis and peritonitis. Examples of diseases and disorders related to Aspergillus infection include, but are not limited to, aspergillosis, chronic lung irritation, hypersensitivity pneumonia, allergic bronchopulmonary aspergillosis, aspergilloma, tracheobronchitis, acute necrotising aspergillus pneumonia, chronic necrotising aspergillus pneumonia and granulomatous aspergillosis.

In accordance with a further embodiment of the invention, one or more compounds of the invention may be used in combination with one or more known anti-fungal agents in combination or synergistic therapy for the treatment of fungal infection, or disorders or diseases associated therewith. The compounds of the invention can be administered before, during or after treatment with the known anti-fungal agent(s). Such combination therapy is known in the art and selection of the appropriate anti-fungal agent(s) to be administered with the compounds of the invention is readily discernible by one of skill in the art. For example, for the treatment of fungal infections and fungally-related diseases, known anti-fungal compounds include, but are not limited to, amphotericin B and the structurally related compounds nystatin and pimaricin; flucytosine; azole derivatives such as ketoconazole, clotrimazole, miconazole, econazole, butoconazole, oxiconazole, sulconazole, terconazole, fluconazole and itraconazole; allylamines-thiocarbamates, such as tolnaftate and naftifme, and griseofulvin.

The invention also contemplates the use of compounds of the invention as the active ingredient in anti-fungal compositions for non-therapeutic uses including, for example, anti-fungal cleansers, polishes, paints, sprays, soaps, and detergents. The compounds of the invention can also be included as an anti-fungal agent in cosmetic, personal care, household and industrial products, for example, to improve shelf-life by inhibiting the growth of fungi within the products. The compounds may be formulated for application to surfaces to inhibit the growth of a fungal species thereon, for example, surfaces such as countertops, desks, chairs, laboratory benches, tables, floors, sinks, showers, toilets, bathtubs, bed stands, tools or equipment, doorknobs and windows. Alternatively, the compounds may be formulated for laundry applications, for example, for washing clothes, towels, sheets and other bed linen, washcloths or other cleaning articles. The anti-fungal cleansers, polishes, paints, sprays, soaps, and detergents comprising the compounds of the invention can optionally contain suitable solvent(s), carrier(s), thickeners, pigments, fragrances, deodorisers, emulsifiers, surfactants, wetting agents, waxes, or oils, as required for the formulation of such products as is known in the art. The cleansers, polishes, paints, sprays, soaps, and detergents comprising the compounds of the invention are useful in institutions, such as in hospital settings, for the prevention of nosocomial infections, as well as in home settings. In one embodiment, the invention provides a formulation containing one or more compounds of the invention for external use as a pharmaceutically acceptable skin cleanser.

In addition, in one embodiment, the invention contemplates the use of compounds of the invention in formulations to inhibit the growth of fungal species in food preparations. In another embodiment, the invention contemplates the use of compounds of the invention in formulations to sterilise surgical and other medical equipment and implantable devices, including prosthetic joints. The compounds can also be formulated for use in the in situ sterilisation of indwelling invasive devices such as intravenous lines and catheters, which are often foci of infection.

In another embodiment, the invention contemplates the use of the compounds of Formula I as the active ingredient in personal care items, such as soaps, deodorants, shampoos, mouthwashes, toothpastes, and the like. Many compositions used in personal care applications are susceptible to fungal growth and it is thus desirable to incorporate into these compositions an effective anti-fungal agent. The anti-fungal agent may be incorporated into the personal care formulation using techniques known in the art. Thus, the anti-fungal agent may be added to the personal care formulation as a solution, emulsion or dispersion in a suitable liquid medium. Alternatively, the anti-fungal agent may be added, undiluted, to the personal care formulation or may be added with a suitable solid carrier or diluent. The anti-fungal agent may be added to the pre-formed personal care formulation or may be added during the formation of the personal care formulation, either separately or premixed with one of the other components of the formulation.

VI. Pharmaceutical Formulations and Therapeutic Administration of Anti-Fungal Compounds of the Invention

For use as therapeutic agents in the treatment of fungal infections, or disorders or diseases associated therewith in a subject, the anti-fungal compounds of the invention are typically formulated prior to administration. Therefore, the invention provides pharmaceutical formulations comprising one or more compounds of the invention and a pharmaceutically-acceptable carrier, diluent, or excipient. The pharmaceutical formulations can be prepared by standard procedures using well-known and readily available ingredients. In making the compositions of the invention, the active ingredient(s) may be mixed with a carrier, or diluted by a carrier, or enclosed within a carrier, and may be in the form of a capsule, sachet, paper, or other container. The carrier may also serve as a diluent and may be a solid, semi-solid, or liquid material.

The pharmaceutical compositions comprising the anti-fungal compounds according to the invention may be administered in a number of ways depending upon whether local or systemic treatment is desired and upon the area to be treated. Administration may be topical (including ophthalmic and to mucous membranes including vaginal and rectal delivery), pulmonary, e.g. by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal or intramuscular injection or infusion; or intracranial, e.g. intrathecal or intraventricular, administration.

The anti-fungal compounds of the invention may be delivered alone or in combination with other anti-fungal agents, and may be delivered along with a pharmaceutically acceptable vehicle. Ideally, such a vehicle would enhance the stability and/or delivery properties. The invention thus provides for administration of pharmaceutical compositions comprising one or more of the compounds of the invention using a suitable vehicle, such as an artificial membrane vesicle (including a liposome, niosome and the like), microparticle or microcapsule. The use of such vehicles may be beneficial, for example, in achieving sustained release of the anti-fungal compound(s).

For administration to an individual for the treatment of an infection or disease, the invention also contemplates the formulation of the pharmaceutical compositions comprising the anti-fungal compounds into oral dosage forms such as tablets, capsules and the like. For this purpose, the compounds can be combined with conventional carriers, such as magnesium carbonate, magnesium stearate, talc, sugar, lactose, pectin, dextrin, starch, gelatin, tragacanth, methylcellulose, sodium carboxymethyl-cellulose, low melting wax, cocoa butter and the like. Diluents, flavouring agents, solubilizers, lubricants, suspending agents, binders, tablet-disintegrating agents and the like can also be employed, if required. The anti-fungal compounds can be encapsulated with or without other carriers. In accordance with the invention, the proportion of anti-fungal compound(s) in any solid and liquid composition will be at least sufficient to impart the desired activity to the individual being treated upon oral administration. The invention further contemplates parenteral injection of the anti-fungal compounds, in which case the compounds are formulated as a sterile solution containing other solutes, for example, enough saline or glucose to make the solution isotonic.

For administration by inhalation or insufflation, the anti-fungal compounds can be formulated into an aqueous or partially aqueous solution, which can then be utilized in the form of an aerosol. Aqueous formulations of the anti-fungal compounds of the invention may also be used in the form of ear or eye drops, or ophthalmic solutions. The invention further contemplates topical use of the anti-fungal compounds. For this purpose they can be formulated as dusting powders, creams or lotions in pharmaceutically acceptable vehicles, which are applied to affected portions of the skin.

Compositions intended for oral use may be prepared according to procedures known in the art for the manufacture of pharmaceutical compositions and such compositions may further contain one or more sweetening agents, flavouring agents, colouring agents, preserving agents, or a combination thereof, in order to provide pharmaceutically elegant and palatable preparations. Tablets typically contain the anti-fungal compound(s) in admixture with non-toxic pharmaceutically acceptable excipients suitable for the manufacture of tablets, such as inert diluents, for example, calcium carbonate, sodium carbonate, lactose, calcium phosphate or sodium phosphate; granulating and disintegrating agents, for example, corn starch, or alginic acid; binding agents, for example, starch, gelatine or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the anti-fungal compound(s) is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Aqueous suspensions typically contain the anti-fungal compound(s) in admixture with excipients suitable for the manufacture of aqueous suspensions, such as suspending agents (for example, sodium carboxylmethylcellulose, methyl cellulose, hydropropylmethyl cellulose, sodium alginate, polyvinylpyrrolidone, gum tragacanth and gum acacia); dispersing or wetting agents such as a naturally-occurring phosphatide (for example, lecithin), or condensation products of an alkylene oxide with fatty acids (for example, polyoxyethylene stearate), or condensation products of ethylene oxide with long chain aliphatic alcohols (for example, hepta-decaethyleneoxycetanol), or condensation products of ethylene oxide with partial esters derived from fatty acids and a hexitol (for example, polyoxyethylene sorbitol monooleate), or condensation products of ethylene oxide with partial esters derived from fatty acids and hexitol anhydrides (for example, polyethylene sorbitan monooleate). The aqueous suspensions may further contain one or more preservatives, for example, ethyl, or n-propyl-p-hydroxy benzoate; one or more colouring agents; one or more flavouring agents, or one or more sweetening agents, such as sucrose or saccharin, or a combination thereof.

Oily suspensions may be formulated by suspending the anti-fungal compound(s) in a vegetable oil, for example, peanut oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavouring agents may be added to provide palatable oral preparations. These compositions may be preserved by the addition of an anti-oxidant such as ascorbic acid.

Dispersible powders and granules suitable for preparation of an aqueous suspension by the addition of water provide the anti-fungal compound in admixture with a dispersing or wetting agent, suspending agent and one or more preservatives. Suitable dispersing or wetting agents and suspending agents are exemplified by those mentioned above. Additional excipients, for example, sweetening, flavouring and colouring agents, may also be present.

Pharmaceutical compositions of the invention may also be in the form of oil-in-water emulsions. The oil phase may be a vegetable oil, for example, olive oil or peanut oil, or a mineral oil, for example, liquid paraffin, or mixtures thereof. Suitable emulsifying agents may be naturally-occurring gums (for example, gum acacia or gum tragacanth); naturally-occurring phosphatides (for example, soy bean lecithin), and esters or partial esters derived from fatty acids and hexitol anhydrides (for example, sorbitan monooleate), and condensation products of the partial esters with ethylene oxide (for example, polyoxyethylene sorbitan monooleate). The emulsions may also contain sweetening and flavouring agents.

Syrups and elixirs may be formulated with sweetening agents, for example, glycerol, propylene glycol, sorbitol or sucrose. Such formulations may also contain one or more demulcents, preservatives or flavouring and colouring agents, or combinations thereof.

The pharmaceutical compositions may be in the form of a sterile injectable aqueous or oleaginous suspension. This suspension may be formulated according to known art using suitable dispersing or wetting agents and suspending agents as described above. The sterile injectable preparation may also be a solution or a suspension in a non-toxic, parenterally acceptable diluent or solvent, for example, as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. Typically, a bland fixed oil is employed for this purpose such as a synthetic mono- or diglyceride. In addition, fatty acids such as oleic acid find use in the preparation of injectables. Adjuvants, local anaesthetics, preservatives and/or buffering agents, may also be included in the injectable formulation.

The one or more compounds of the invention may be administered, together or separately, in the form of suppositories for rectal or vaginal administration of the compound. These compositions can be prepared by mixing the compound with a suitable non-irritating excipient which is solid at ordinary temperatures but liquid at the rectal/vaginal temperature and will therefore melt to release the compound. Examples of such materials include cocoa butter and polyethylene glycols.

Another formulation of the invention employs transdermal delivery devices (“patches”). Such transdermal patches may be used to provide continuous or discontinuous infusion of the anti-fungal compounds of the invention in controlled amounts. The construction and use of transdermal patches for the delivery of pharmaceutical agents is well known in the art (see, for example, U.S. Pat. No. 5,023,252; issued Jun. 11, 1991). Such patches may be constructed for continuous, pulsatile, or on demand delivery of pharmaceutical agents.

It may be desirable or necessary to introduce the pharmaceutical composition to the brain, either directly or indirectly. Direct techniques usually involve placement of a drug delivery catheter into the host's ventricular system to bypass the blood-brain barrier. An example of such an implantable delivery system, used for the transport of biological factors to specific anatomical regions of the body, is described in U.S. Pat. No. 5,011,472.

The dosage of the anti-fungal compound to be administered is not subject to defined limits, but will usually be an effective amount. In general, the dosage will be the equivalent, on a molar basis, of the pharmacologically active free form produced from a dosage formulation upon the metabolic release of the active free drug to achieve its desired pharmacological and physiological effects. The pharmaceutical compositions are typically formulated in a unit dosage form, each dosage containing from, for example, about 0.01 to about 100 mg of the anti-fungal compound. The term “unit dosage form” refers to physically discrete units suitable as unitary dosages for administration to human subjects and other animals, each unit containing a predetermined quantity of anti-fungal compound calculated to produce the desired therapeutic effect, in association with a suitable pharmaceutical excipient.

Typical daily dosages of the anti-fungal compounds fall within the range of about 0.01 to about 200 mg/kg of body weight in single or divided dose. However, it will be understood that the amount of the compound actually administered will be determined by a physician, in the light of the relevant circumstances, including the condition to be treated, the chosen route of administration, the actual compound administered, the age, weight, and response of the individual patient, and the severity of the patient's symptoms, and therefore the above dosage ranges are not intended to limit the scope of the invention in any way. In some instances dosage levels below the lower limit of the aforesaid range may be adequate, while in other cases still larger doses may be employed without causing any harmful side effect, for example, by first dividing larger doses into several smaller doses for administration throughout the day.

VII. Kits

The invention additionally provides for therapeutic kits containing one or more compounds of the invention in pharmaceutical compositions or unit dosage forms, alone or in combination with one or more other anti-fungal agents, for use in the treatment of fungal infections, or fungally-related diseases or disorders. Individual components of the kit can be packaged in separate containers and, associated with such containers, can be a notice in the form prescribed by a governmental agency regulating the manufacture, use or sale of pharmaceuticals or biological products, which notice reflects approval by the agency of manufacture, use or sale for human or animal administration. The kit can optionally further contain one or more other anti-fungal agents for use in combination with the compound(s) of the invention. The kit may optionally contain instructions or directions outlining the method of use or dosing regimen for the compound(s) and/or additional anti-fungal agents.

When the components ofthe kit are provided in one or more solutions, the solution can be an aqueous solution, for example a sterile aqueous solution. In this case the container means may itself be an inhalant, syringe, pipette, eye dropper, or other such like apparatus, from which the solution may be administered to a subject or applied to and mixed with the other components of the kit.

The components of the kit may also be provided in dried or lyophilised forms. When reagents or components are provided as a dried form, reconstitution generally is by the addition of a suitable solvent. It is envisioned that the solvent also may be provided in another container means. Irrespective of the number or type of containers, the kits of the invention also may comprise, or be packaged with, an instrument for assisting with the administration of the final composition or unit dosage form to a patient. Such an instrument may be an inhalant, syringe, pipette, forceps, measured spoon, eye dropper or similar medically approved delivery vehicle.

To gain a better understanding of the invention described herein, the following examples are set forth. It will be understood that these examples are intended to describe illustrative embodiments of the invention and are not intended to limit the scope of the invention in any way.

### Examples

Preparation of Compounds:

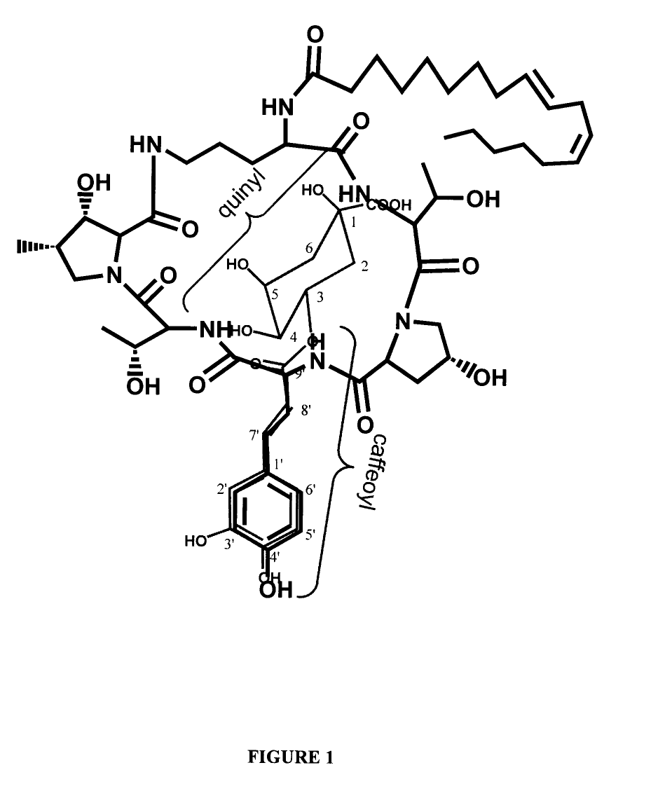

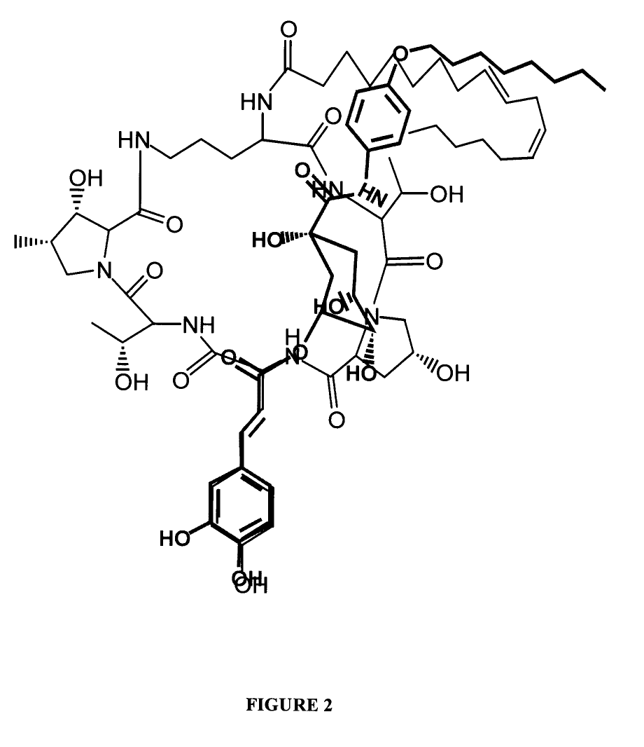

For echinocandin compounds, the position of homotyrosine ring with respect to the lipophilic side chain is believed to be a contributing factor for the antifungal activity of these compounds (Zambias, R. A., et aL, ibid.; Ma, C-H., et al., ibid.). A new series of compounds were therefore designed using chlorogenic acid as the core building block due to its partial structural similarity to that of the homotyrosine and hydroxyproline components of the “southeastern” part of echinocandin B (see FIG. 1). Superimposition of the structure of one of these compounds (compound 2, see Example 2 below) on the structure of echinocandin is shown in FIG. 2.

Compounds 1 to 23 were prepared as described below and characterized by NMR and MS spectra. ¹H-NMR was performed in a 500 MHz Brucker instrument at room temperature using a suitable deuterated solvent.

### Example 1

### Preparation of Compound 1

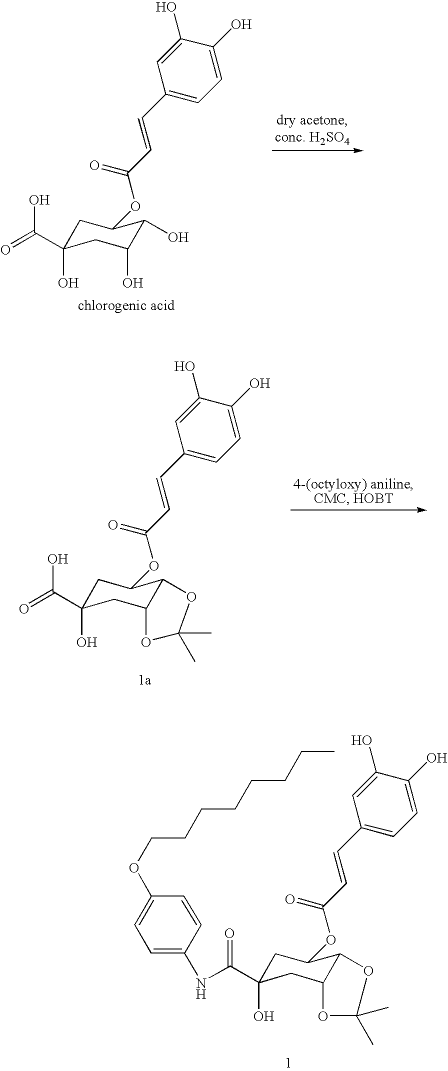

Chlorogenic acid (1 mmol) was suspended in dried acetone (7 ml). The mixture was cooled in ice-H₂O and conc. H₂SO₄ (0.059 ml) was added. The solution mixture was stirred overnight at room temperature (r.t.), neutralized with Na₂CO₃ (0.609 g) to pH=5-6, filtered off, and concentrated to dryness to obtain chlorogenic acid acetonide derivative 1a as a white powder.

To a DMF solution (15 ml) of acetonide 1a and 4-(octyloxy) aniline (1 mmol each) was added CMC (1.5 mmol) and HOBT (1.5 mmol). The reaction mixture was stirred at r.t. overnight, filtered, concentrated to dryness and chromatographed on ODS to afford compound 1 by elution with 80-90% MeOH.

Compound 1a: white solid (400 mg, 100%), 4,5-chlorogenic acid actonide containing about 5% of 1,1,4,5-chlorogenic acid diacetonide. ¹H NMR (CD₃OD, 500 MHz), δ 1.31 (s, 3H), 1.49 (s, 3H) (4,5-acetonide), 1.94 (t, J=11.5 Hz, 1H), 2.03 (dd, J=13.0, 3.5 Hz, 1H), 2.15 (br.s, 1H), 2.33 (dd, J=16.0, 5.0 Hz, 1H), 4.16 (t, J=7.0 Hz, 1H), 4.50 (br.s, 1H), 5.43 (m, 1H), 6.24 (d, J=15.5 Hz, 1H), 6.77 (d, J=8.0 Hz, 1H), 6.93 (br.d, J=8.0 Hz, 1H), 7.04 (br.s, 1H), 7.53 (d, J=15.5 Hz, 1H). ESI-MS (Negative): 393.3([M−H]⁻, 100%); ESI-MS (Positive): 395.1 ([M+H]⁺, 100%), 162.6 (30%)

Compound 1: white solid (280 mg from one mole of starting material, 46.8%). ¹H NMR (CDCl₃, 500 MHz), δ 0.88 (t, J=7.0 Hz, 3H), 1.31 (m, 8H), 1.40 (s, 3H), 1.45 (m, 2H), 1.63 (s, 3H), 1.75 (m, 2H), 2.12 (t. J=12.0 Hz, 1H), 2.24 (br.d, J=11.0 Hz, 1H), 2.31 (d, J=16.0 Hz, 1H), 2.54 (br.d, J=13.0 Hz, 1H), 3.92 (t, J=6.0 Hz, 2H), 4.28 (br.s, 1H), 4.61 (br.s, 1H), 5.46 (m, 1H), 6.19 (d,J=16.0 Hz, 1H), 6.86 (m, 3H), 6.90 (d, J=7.5 Hz, 1H), 7.05 (br.s, 1H), 7.44 (d, J=9.0 Hz, 2H), 7.54 (d, J=16.0 Hz, 1H), 8.84 (br.s, 1H). APCI-MS (Negative): 596.4 ([M−H]⁻, 100%); ESI-MS (Positive): 620.2 ([M+Na]⁺, 35%).

### Example 2

### Preparation of Compound 2

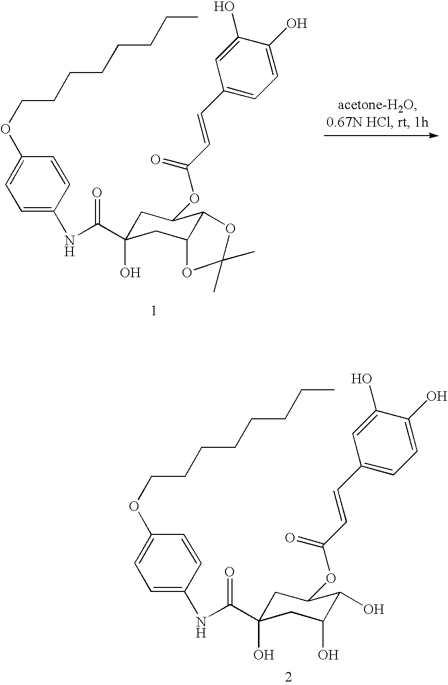

Compound 1 was dissolved in acetone (6 ml) and 1N HCl (4 ml). The mixture was stirred at r.t. for 1 h, neutralized with 1N NaOH to pH=6, concentrated to dryness and passed through an ODS column to obtain compound 2 by elution with 80% MeOH.

Compound 2: white solid (120 mg 2 from 160 mg 1, 80.4%). ¹H NMR (CDCl₃+CD₃OD, 500 MHz), δ 0.88 (t, J=7.0 Hz, 3H), 1.32 (m, 8H), 1.44 (m, 2H), 1.76 (m, 2H), 2.23 (m, 4H), 3.79 (dd, J=9.5, 2.0 Hz, 1H), 3.93 (t, J=7.0 Hz, 2H), 4.33 (br.s, 1H), 5.41 (m, 1H), 6.18 (d, J=15.5 Hz, 1H), 6.80 (d, J=7.5 Hz, 1H), 6.86 (m, 3H), 7.03 (br.s, 1H), 7.45 (d, J=8.5 Hz, 2H), 7.53 (d, J=15.5 Hz, 1H). ESI-MS (Negative): 556.3 ([M−H]⁻, 100%); ESIMS (Positive): 580.2 ([M+Na]⁺, 100%). Negative HR-FAB-MS: 556.2516 ([M−H]⁻, C₃₀H₃₈O₉N⁻; Calc. 556.2546).

### Example 3

### Preparation of Compound 3

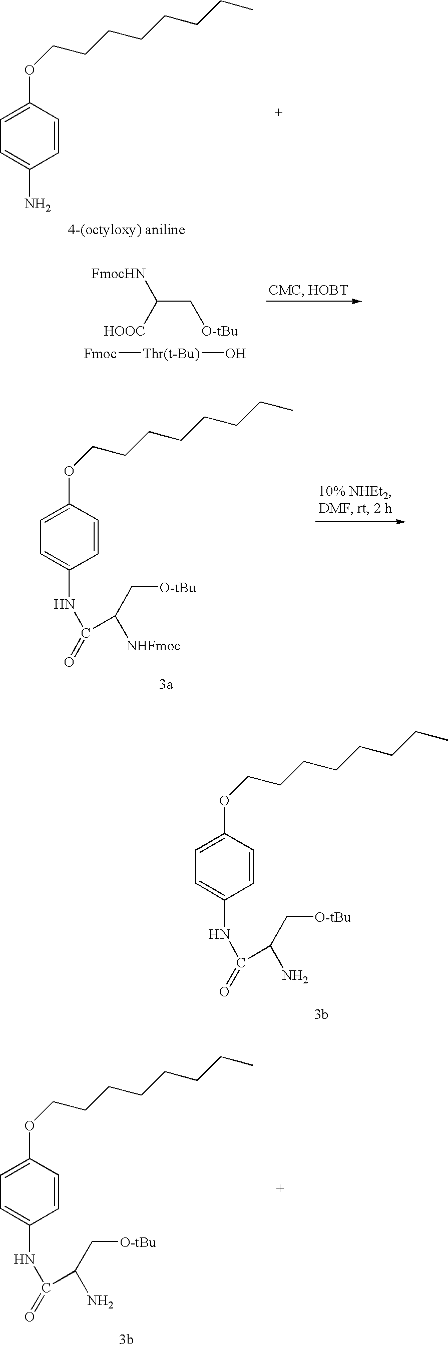

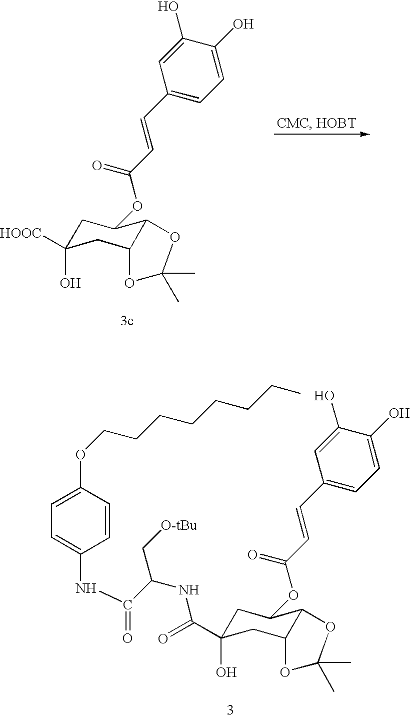

Fmoc-Thr(t-Bu)-OH (2 mmol) in DMF (15 ml) was condensed with 4-(octyloxy)aniline (2 mmol) in the presence of CMC (3 mmol) and HOBT (3 mmol) to obtain the amino acid derivative 3a with a lipophilic chain. Compound 3a was deprotected with diethylamine (1.5 ml) in DMF (15 ml) at r.t. for 2 h, then concentrated to dryness at 70° C. to obtain compound 3b (i.e., H₂N-aa-4-(octyloxy)aniline) with the free amino group. Compound 3b (1 mmol) was reacted at r.t. overnight with the acetonide of chlorogenic acid 3c (1 mmol) in 15 ml DMF, in the presence of CMC (1.5 mmol) and HOBt (1.5 mmol). The reaction mixture was filtered, concentrated to dryness and chromatographed in ODS to obtain compound 3 by elution with 80-90% MeOH.

Compound 3: white solid (400 mg, 54%). ¹H NMR (CDCl₃+CD₃OD, 500 MHz), 8 0.89 (t, J=7.0 Hz, 3H), 1.28 (m, 17H), 1.38 (s, 3H), 1.41 (m, 2H), 1.60 (s, 3H), 1.62 (m, 2H), 1.78 (m, 2H), 2.07 (t, J=12.0 Hz, 1H), 2.20 (br.d, J=9.0 Hz, 1H), 2.30 (d, J=16.0 Hz, 1H), 2.45 (d, J=12.5 Hz, 1H), 3.49 (t, J=8.0 Hz, 1H), 3.78 (m, 1H), 3.93 (t, J=7.0 Hz, 2H), 4.08 (br.s, 1H), 4.23 (m, 1H), 4.54 (m, 1H), 4.59 (br.s, 1H), 5.43 (m, 1H), 6.20 (d, J=15.5 Hz, 1H), 6.81(m, 3H), 6.90 (d, J=8.5 Hz, 1H), 7.05 (d, J=2.0 Hz, 1H), 7.39 (d, J=8.5 Hz, 2H), 7.58 (d, J=15.5 Hz, 1H), 8.04 (d, J=8.5 Hz, 1H, NH), 8.58 (s, 1H, NH). API-ESMS (Negative): 739.3 ([M−1]⁻, 100%); API-ESMS (Positive): 763.3 ([M+Na]⁺, 18%).

### Examples 4, 5 and 6

### Preparation of Compounds 4, 5, and 6

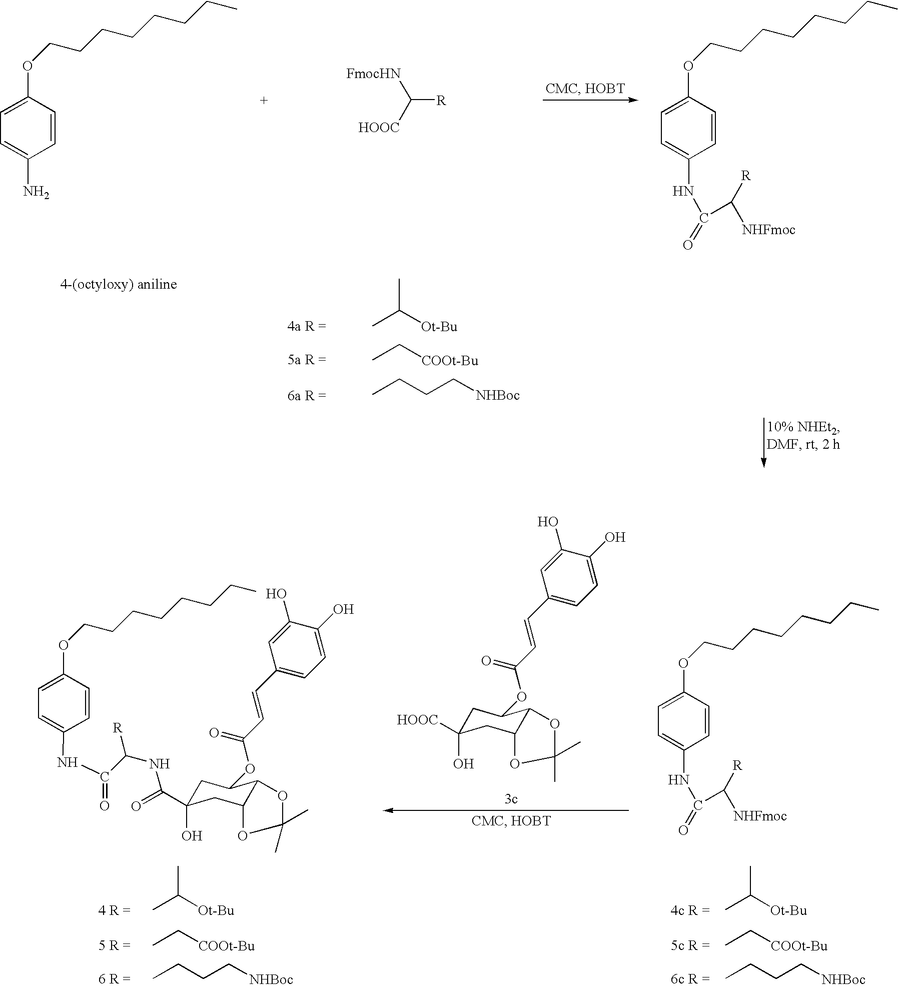

Compounds 4, 5 and 6 were also prepared following the synthetic procedure as described for compound 3, i.e., by condensing the appropriate starting amino acid derivative [i.e., Fmoc-Ser(tBu)-OH 4a, Fmoc-Asp(OtBu)-OH 5a or Fmoc-Orn(Boc)-OH) 6a, respectively] with 4-(octyloxy)aniline in the presence of CMC and HOBT to obtain respective amino acid derivative 4b, 5b and 6b with a lipophilic chain. Compounds 4b, 5b and 6b were individually de-protected with NHEt₂ to obtain compounds 4c, 5c and 6c, respectively. Compounds 4c, 5c and 6c were individually reacted with the acetonide of chlorogenic acid 3c to obtain compounds 4, 5 and 6, respectively.

Compound 4: white solid (192 mg, 25.4%).¹H NMR (CDCl₃, 500 MHz), δ 0.88 (t, J=7.0 Hz, 3H), 1.08 (d, J=6.5 Hz, 3H) 1.30 (m, 21H), 1.40 (m, 2H), 1.60 (m, 2H), 1.78 (m, 2H), 2.02 (m, 1H), 2.19 (m, 1H), 2.30 (m, 1H), 2.41 (m, 1H), 3.92 (t, J=7.0 Hz, 2H), 4.18 (m, 2H), 4.48 (m, 2H), 5.44 (m, 1H), 6.17 (d, J=15.5 Hz, 1H), 6.86 (m, 4H), 7.02 (br.s, 1H), 7.38 (d, J=9.0 Hz, 2H), 7.53 (d, J=15.5 Hz, 1H), 8.22 (d, J=6.5 Hz, 1H), 9.05 (s, 1H). ESI-MS (Negative): 790.0 ([M+Cl]⁻, 100%), 753.8 ([M−H]⁻, 20%).

Compound 5: white solid (40.3%). ¹H NMR (CDCl₃, 500 MHz), δ 0.88 (t, J=7.0 Hz, 3H), 1.34 (m, 11H), 1.46 (m, 12H), 1.59 (m, 2H), 1.75 (m, 2H), 2.07 (t, J=12.0 Hz, 1H), 2.17 (br.d, J=9.0 Hz, 1H), 2.24 (d, J=16.0 Hz, 1H), 2.45 (d, J=12.5 Hz, 1H), 2.72 (dd, J=7.5, 16.0 Hz, 1H), 2.84 (br.d, J=16.5 Hz, 1H), 3.90 (t, J=7.0 Hz, 2H), 4.26 (t, J=6.0 Hz, 1H), 4.56 (br.s, 1H), 4.81 (br.d, J=4.5 Hz, 1H), 5.41 (m, 1H), 6.18 (d, J=15.5 Hz, 1H), 6.82 (m, 3H), 6.89 (br.s, 1H), 7.06 (br.s, 1H), 7.35 (d, J=8.0 Hz, 2H), 7.53 (d, J=15.5 Hz, 1H), 8.30 (d, J=8.5 Hz, 1H), 8.58 (br.s, 1H). ESIMS (Negative): 767.4 ([M−H]⁻, 100%).

Compound 6: white solid (430 mg, 53.0%). ¹H NMR (CDCl₃, 500 MHz), δ 0.88 (t, J=7.0 Hz, 3H), 1.36 (m, 21H), 1.54 (m, 1H), 1.58 (s, 3H), 1.75 (m, 3H), 1.93 (m, 1H), 2.07 (t.J=12.0 Hz, 1H), 2.16 (m, 1H), 2.24 (d, J=15.0 Hz, 1H), 2.41 (d, J=13.0 Hz, 1H), 3.03 (m, 1H), 3.40 (m, 1H), 3.91 (t, J=6.0 Hz, 2H), 4.26 (t, J=6.5 Hz, 1H), 4.53 (br.s, 1H), 4.75 (br.s, 1H), 4.96 (br.s, 1H), 5.42 (m, 1H), 6.17 (d, J=16.0 Hz, 1H), 6.83 (m, 4H), 7.06 (br.s, 1H), 7.39 (d, J=8.5 Hz, 2H), 7.53 (d, J=16.0 Hz, 1H), 7.84 (d, J=8.0 Hz, 1H), 8.78 (br.s, 1H). APCI-MS (Negative): 810.5 ([M−H]⁻, 100%); ESI-MS (Positive): 834.3 ([M+Na]⁺, 10%), 498.3 (100%).

### Examples 7, 8, 9 and 10

### Preparation of Compounds 7, 8, 9 and 10

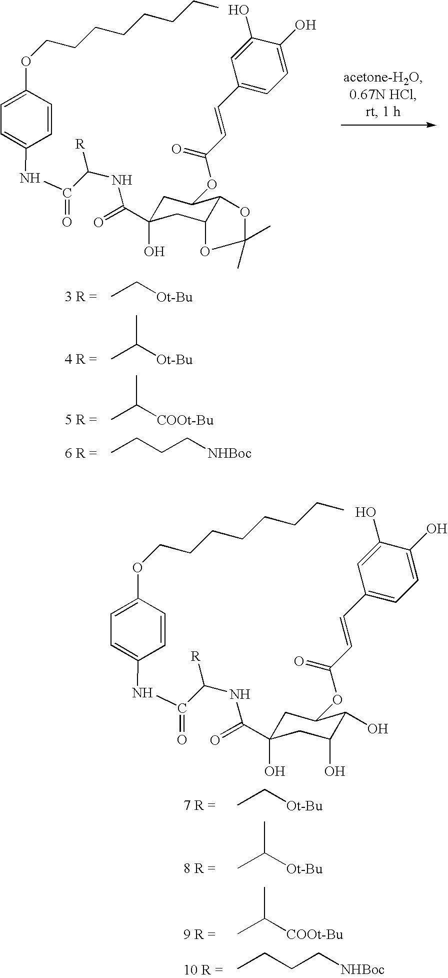

Compounds 7, 8, 9 and 10 were prepared by acid hydrolysis of compounds 3, 4, 5 and 6, respectively, under controlled conditions as follows. Compound 3, 4, 5 or 6 was dissolved in acetone (6 ml) and 1N HCl (4 ml). The mixture was stirred at r.t. for 1 h, neutralized 1N NaOH to pH=6, concentrated to dryness and passed through an ODS column to afford compound 7, 8, 9 or 10, respectively, by elution with 80% MeOH.

Compound 7: white solid (obtained 270 mg 7 from 300 mg 3, 95%). ¹H NMR (CDCl₃+CD₃OD, 500 MHz), δ 0.89 (t, J=7.0 Hz, 3H), 1.20 (s, 9H), 1.28 (m, 8H), 1.41 (m, 2H), 1.75 (m, 2H), 2.09 (m, 3H), 2.23 (m, 1H), 3.49 (t, J=8.0 Hz, 1H), 3.73 (m, 1H), 3.80 (d, J=8.5 Hz, 1H), 3.93 (t, J=7.0 Hz, 2H), 4.31 (br.s, 1H), 4.54 (m, 1H), 5.43 (m, 1H), 6.03 (d, J=15.5 Hz, 1H), 6.74(m, 2H), 6.83 (d, J=8.5 Hz, 2H), 6.96 (d, J=2.0 Hz, 1H), 7.37 (d, J=8.5 Hz, 2H), 7.40 (d, J=15.5 Hz, 1H), 8.04 (d, J=8.5 Hz, 1H, NH). ESI\MS (Negative): 699.5 ([M−H]⁻, 100%); ESI-MS (Positive): 723.2 ([M+Na]⁺, 50%).

Compound 8: white solid (147 mg, 80%). ¹H NMR (CDCl₃+CD₃OD, 500 MHz), δ 0.87 (t, J=7.0 Hz, 3H), 1.16 (d, J=5.0 Hz, 3H), 1.28 (m, 18H), 1.44 (m, 2H), 1.79 (m, 2H), 2.15 (m, 2H), 2.25 (d, J=10.0 Hz, 1H), 3.76 (br.s, 1H), 3.96 (t, J=7.0 Hz, 2H), 4.20 (m, 1H), 4.45 (m, 1H), 5.42 (br.s, 1H), 6.22 (d, J=14.5 Hz, 1H), 6.88 (overlapped signals., 3H), 6.94 (d, J=7.0 Hz, 1H), 7.04 (d, J=7.0 Hz, 1H), 7.39 (d, J=9.0 Hz, 2H), 7.66 (d, J=14.5 Hz, 1H). Positive FAB-MS m/z 715.4 ([M+H]⁺, 1%); 737.4 ([M+Na]⁺, 13%); Negative FAB-MS m/z 713.4 ([M−H]⁻, 100%); Negative HR-FAB-MS m/z 713.3633 ([M−H]⁻, C₃₈H₅₃N₂O₁₁⁻, Calc. 713.3649).

Compound 9: white solid (60.0%). ¹H NMR (CDCl₃+CD₃OD, 500 MHz), δ 0.88 (t, J=7.0 Hz, 3H), 1.29 (m, 8H), 1.40 (m, 2H), 1.44 (s, 9H), 1.75 (m, 2H), 2.11 (m, 3H), 2.25 (NMR solvent signals, possibly overlapped with compound signals), 2.77 (m, 2H), 3.74 (dd, J=9.5, 2.5 Hz, 1H), 3.90 (t, J=7.0 Hz, 2H), 4.27 (d, J=2.0 Hz, 1H), 4.80 (m, 1H), 5.38 (m, 1H), 6.18 (d, J=15.5 Hz, 1H), 6.83 (m, 4H), 7.02 (br. s, 1H), 7.37 (d, J=8.0 Hz, 2H), 7.51 (d, J=15.5 Hz, 1H). ESI-MS (Negative): 727.5 ([M−H]⁻, 100%); ESI-MS (Positive): 751.3 ([M+Na]⁺, 25%), 415.2 (100%).

Compound 10: purified by HPLC, white solid (200 mg from 400 mg of 6, 52.6%). ¹H NMR (CDCl₃+CD₃OD, 500 MHz), δ 0.88 (t, J=7.0 Hz, 3H), 1.29 (m, 8H), 1.41 (m, 10H), 1.51 (br.s, 1H), 1.76 (m, 3H), 1.89 (m, 1H), 2.11 (m, 3H), 2.21 (m, 3H), 3.06 (m, 1H), 3.22 (m, 1H), 3.75 (br.d, J=9.5, 1H), 3.91 (t, J=7.0 Hz, 2H), 4.25 (br.s, 1H), 4.59 (br.s, 1H), 5.41 (m, 1H), 6.18 (d, J=16.0 Hz, 1H), 6.82 (m, 4H), 7.05 (br.s, 1H), 7.42 (d, J=8.5 Hz, 2H), 7.51 (d, J=16.0 Hz, 1H). APCI-MS (Negative): 770.4 ([M−H]⁻, 100%); ESI-MS (Positive): 794.4 ([M+Na]⁺, 100%).

### Examples 11, 12, 13 and 14

### Preparation of Compounds 11, 12, 3 and 14

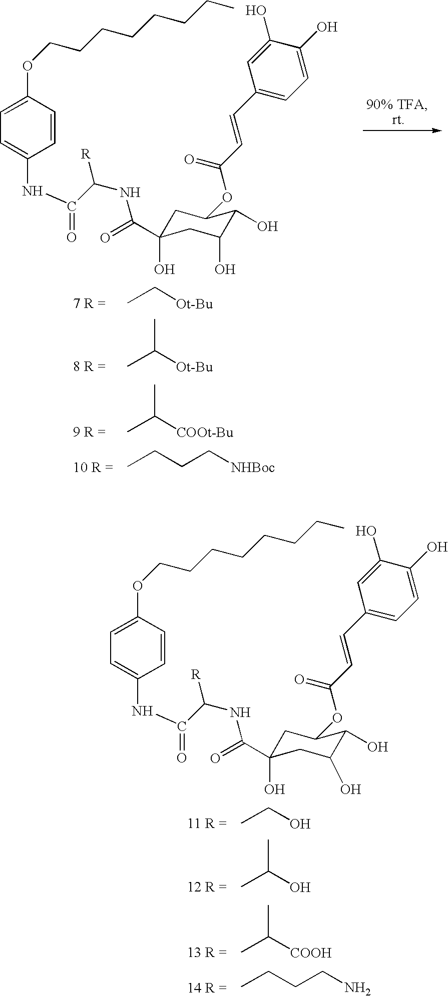

Compounds 11, 12, 13, and 14 were prepared by reacting compounds 7, 8, 9, and 10, respectively, with 90% TFA under controlled acid concentration at r.t. for 30 min. to avoid the hydrolysis of the ester bond between caffeoyl and quinic acid groups. The reaction mixture was concentrated to dryness, and passed through a SiO₂ column to obtain compound 11, 12, 13 or 14 as a white powder by elution with CHCl₃-MeOH (15:2-9:1).

Compound 11: white solid (from 130 mg 7, 90%). ¹H NMR (CDCl₃+CD₃OD, 500 MHz), δ 0.87 (t, J=7.0 Hz, 3H), 1.28 (m, 8H), 1.44 (m, 2H), 1.75 (m, 2H), 2.09 (m, 3H), 2.23 (m, 1H), 3.70 (NMR solvent signals, possibly overlapped with compound signals), 3.89 (m, 3H), 4.26 (m. 1H), 4.48 (m, 1H), 5.38 (m, 1H), 6.25 (d, J=15.5 Hz, 1H), 6.79 (d, J=7.5 Hz, 1H), 6.83 (d, J=8.5 Hz, 2H), 6.93 (dd, J=7.5, 2.0 Hz, 1H), 7.05 (d, J=2.0 Hz, 1H), 7.42 (d, J=8.5 Hz, 2H), 7.58 (d, J=15.5 Hz, 1H), 8.05 (d, J=8.5 Hz, 1H, NH). ESI-MS (Negative): 757.3 ([M+TFA]⁻, 100%), 643.3 ([M−1]⁻, 32%); ESI-MS (Positive): 667.2 ([M+Na]⁺, 80%), 645.2 ([M+H]⁺, 100%). Negative HR-FAB-MS: 643.2879 ([M−H]⁻, C₃₃H₄₃O₁₁N₂; Calc. 643.2867).

Compound 12: white solid (50 mg, 90%). ¹H NMR (CD₃OD, 500 MHz)), δ 0.92 (t, J=7.0 Hz, 3H), 1.21 (d, J=6.5 Hz, 3H), 1.38 (m, 8H), 1.47 (m, 2H), 1.75 (m, 2H), 2.03 (m, 3H), 2.20 (m, 1H), 3.72 (dd, J=5.0, 10.0 Hz, 1H), 3.93 (t, J=7.0 Hz, 2H), 4.25 (m. 2H), 4.40 (br.s., 1H), 5.44 (m, 1H), 6.31 (d, J=15.5 Hz, 1H), 6.79 (d, J=7.0 Hz, 1H), 6.90 (d, J=9.0 Hz, 2H), 6.96 (dd, J=2.0, 7.0 Hz, 1H), 7.08 (d, J=2.0 Hz, 1H), 7.43 (d, J=9.0 Hz, 2H), 7.60 (d, J=15.5 Hz, 1H). ESI-MS (Positive): 659.3 ([M+H]⁺, 100%). Positive FAB-MS m/z 659.3 (M+H⁺, 4%); 681.3 ([M+Na]⁺, 8%); Negative FAB-MS m/z 657.3 ([M−H]⁻, 100%); Negative HR-FAB-MS m/z 657.3031 ([M−H]⁻, C₃₄H₄₅N₂O₁₁⁻, Calc. 657.3023).

Compound 13: white solid (60.0%). ¹H NMR (CDCl₃+CD₃OD, 500 MHz), δ 0.87 (t, J=7.0 Hz, 3H), 1.28 (m, 8H), 1.40 (m, 2H), 1.73 (m, 2H), 1.95 (m, 2H), 2.06 (m, 1H), 2.54 (NMR solvent signals, possibly overlapped with compound signals), 2.86 (m, 2H), 3.72 (m, 1H), 3.87 (t, J=7.0 Hz, 2H), 4.22 (br.s. 1H), 4.84 (br.s, 1H), 5.35 (m, 1H), 6.25 (d,J=15.5 Hz, 1H), 6.77 (m, 4H), 6.90 (br.s, 1H), 7.34 (d, J=8.0 Hz, 2H), 7.42 (d, J=15.5 Hz, 1H), 8.24 (d, J=8.5 Hz, 1H, NH). ESI-MS (Negative): 671.4 ([M−H]⁻, 100%); ESIMS (Positive): 695.3 ([M+Na]⁺, 100%). Negative HR-FAB-MS: 671.2820 ([M−H]⁻, C₃₄H₄₃O₁₂N₂⁻; Calc. 671.2816).

Compound 14: white solid (from 41 mg 10, 90%). ¹H NMR (CDCl₃+CD₃OD, 500 MHz), δ 0.87 (t, J=7.0 Hz, 3H), 1.29 (m, 8H), 1.41 (m, 2H), 1.62 (m, 4H), 1.91 (m, 1H), 2.02 (m, 2H), 2.13 (m, 1H), 2.94 (br.s, 2H), 2.38 (NMR solvent signals, possibly overlapped with compound signals), 3.70 (br.d, J=6.0 Hz, 1H), 3.88 (t, J=7.0 Hz, 2H), 4.23 (br.s. 1H), 4.51 (br.s, 1H), 5.36 (m, 1H), 6.08 (d, J=15.5 Hz, 1H), 6.73 (m, 2H), 6.80 (d, J=8.0 Hz, 2H), 6.95 (br.s, 1H), 7.39 (m, 3H). ESI-MS (Negative): 670.5 ([M−H]⁻, 60%); ESIMS (Positive): 672.3 ([M+H]⁺, 100%). Negative HR-FAB-MS: 670.3335 ([M−H]⁻, C₃₅H₄₈O₁₀N₃⁻; Calc. 670.3339).

### Example 15

### Preparation of Compound 15

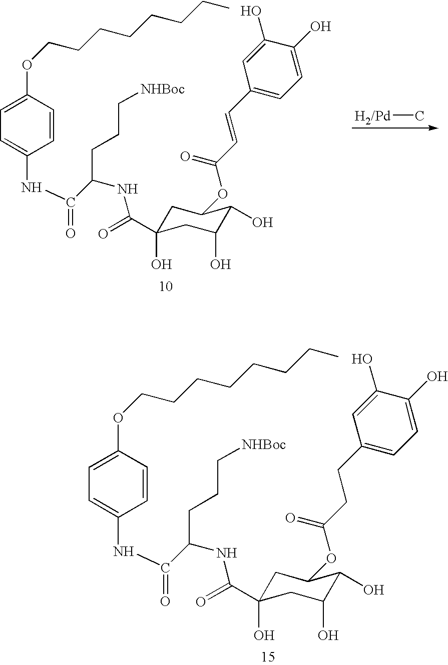

Compound 10 (30 mg, 0.040 mmol) was dissolved in 10 ml of EtOH. To the solution was added 15 mg of Pd/C. The mixture was vigorously stirred under a hydrogen balloon for 15 h. The mixture was filtered and the filtrate was concentrated to dryness to obtain compound 15.

Compound 15: white solid (25 mg, 0.032 mmol, 80%). ¹H NMR (CDCl₃+CD₃OD, 500 MHz), δ 0.89 (t, J=7.0 Hz, 3H), 1.29 (m, 8H), 1.40 (m, 11H), 1.52 (m, 1H), 1.73 (m, 3H), 2.00 (m, overlapped with H₂O signal), 2.60 (br.s. 2H), 2.81 (br.s. 2H), 3.04 (m, 1H), 3.28 (m, 1H), 3.49 (m, 1H), 3.61 (m, 1H), 3.90 (t. J=6.5 Hz, 2H), 4.20 (br.s, 1H), 4.60 (br.s, 1H), 5.34 (m, 1H), 6.59 (d, J=6.0 Hz, 1H), 6.71 (d, J=2.0 Hz, 1H), 6.80 (dd, J=6.0, 2.0 Hz, 1H), 6.89 (d, J=8.0 Hz, 2H), 7.44 (d, J=8.0 Hz, 2H), 8.00 (2H). ESI-MS (Positive): 774.3 ([M+H]⁺, 100%).

### Example 16

### Preparation of Compound 16

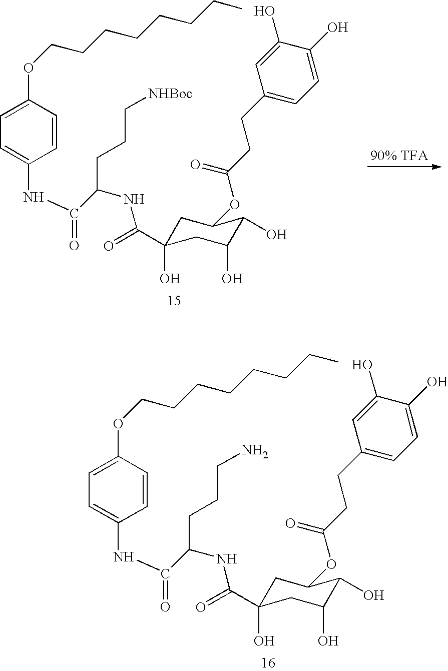

Compound 15 (17 mg, 0.022 mmol) was treated with 90% TFA at r.t. for 30 min and concentrated to dryness to obtain compound 16.

Compound 16: (11 mg, 0.016 mmol, 72.7%). ¹H NMR (CDCl₃+CD₃OD, 500 MHz), δ 0.89 (t, J=7.0 Hz, 3H), 1.30 (m, 8H), 1.42 (m, 2H), 1.75 (m, 6H), 1.93 (m, 2H), 2.61 (m, overlapped with H₂O signal), 2.74 (br.s. 2H), 3.60 (m, 1H), 3.89 (t. J=6.5 Hz, 2H), 4.20 (br.s, 1H), 4.54 (br.s, 1H), 5.21 (m, 1H), 6.49 (d, J=6.0 Hz, 1H), 6.63 (br.s, 1H), 6.71 (br.s, 1H), 6.81 (d, J=9.0 Hz, 2H). 7.41 (d, J=9.0 Hz, 2H). ESI-MS (Negative): 708.9 ([M+Cl]⁻, 100%), 672.7 ([M−H]⁻, 20%). Negative HR-FAB-MS: 672.3499 ([M−H]⁻, C₃₅H₅₀O₁₀N₃⁻; Calc. 672.3496).

### Example 17

### Preparation of Compound 17

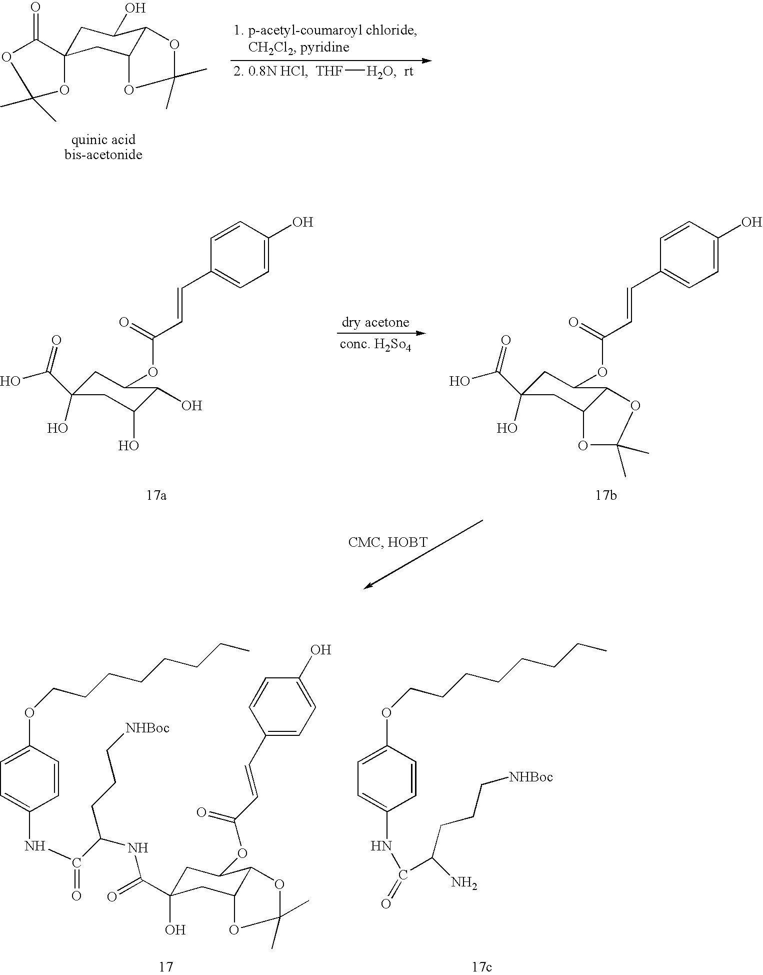

Quinic acid bis-acetonide was condensed with p-acetyl-coumaroyl chloride followed by deprotection of the resulting compound with 0.8N HCl to obtain compound 17a. Compound 17a was treated with dry acetone in the presence of catalytic amount of conc. sulfuric acid to obtain acetonide 17b. Acetonide 17b was condensed with compound 17c to obtain compound 17.

Compound 17a: white solid (950 mg, 77.3%). ¹H NMR (CD₃OD, 500 MHz), δ 2.05 (m, 2H), 2.20 (m, 2H), 3.72 (dd, J=3.5, 7.5 Hz, 1H), 4.17 (m, 1H), 5.35 (m, 1H), 6.32 (d, J=16.0 Hz, 1H), 6.80 (d, J=7.5 Hz, 2H), 7.46 (d, J=7.5 Hz, 2H), 7.62 (d, J=16.0 Hz, 1H). ¹³C NMR (CDCl₃—CD₃OD, 75 MHz), δ 36.7, 38.3, 70.2, 70.5, 73.1, 75.0, 113.7, 115.4, 125.4, 129.6, 145.3, 159.1, 167.5, 175.4. ESI-MS (Negative): 675.3 ([2M−H]⁻, 100%), 336.8 ([M−H]⁻, 40%).

Compound 17: white solid (300 mg, 47.2%). ¹H NMR (CDCl₃, 500 MHz), δ 0.88 (t, J=7.0 Hz, 3H), 1.38 (m, 21H), 1.50 (m, 2H), 1.59 (s, 3H), 1.75 (m, 4H), 1.95 (m, 1H), 2.07 (t.J=12.0 Hz, 1H), 2.19 (dd, J=2.5, 13.5 Hz, 1H), 2.24 (d, J=15.5 Hz, 1H), 2.41 (dd, J=3.0, 15.5 Hz, 1H), 3.01 (m, 1H), 3.41 (m, 1H), 3.93 (t, J=6.0 Hz, 2H), 4.26 (t, J=6.5 Hz, 1H), 4.53 (br.s, 1H), 4.75 (br.s, 1H), 4.82 (br.s, 1H), 5.44 (m, 1H), 6.20 (d, J=16.0 Hz, 1H), 6.83 (m, 4H), 7.32 (d, J=8.5 Hz, 2H), 7.41 (d, J=8.5 Hz, 2H), 7.80 (d, J=16.0 Hz, 1H), 7.78 (d, J=8.5 Hz, 1H), 8.62 (br.s, 1H). APCI-MS (Negative): 794.4 ([M−H]⁻, 100%), 580.3 (45%); APCI-MS (Positive): 796.3 ([M+H]⁺, 10%), 696.3 (100%).

### Example 18

### Preparation of Compound 18

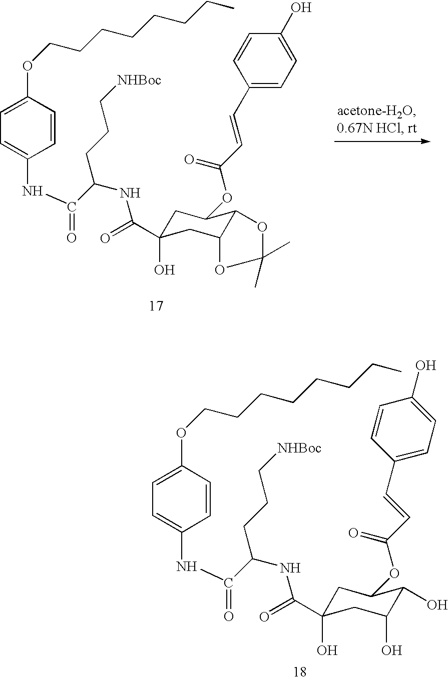

Compound 17 was treated with acetone-H₂O under acidic condition to yield compound 18.

Compound 18: white solid (obtained 120 mg from 150 mg of compound 17, 84.2%). ¹H NMR (CDCl₃+CD₃OD, 500 MHz), δ 0.88 (t, J=7.0 Hz, 3H), 1.29 (m, 8H), 1.41 (m, 11H), 1.51 (m, 2H), 1.73 (m, 3H), 1.89 (m, 1H), 2.18 (m, 7H, possibly containing water signals of the NMR solvent), 2.21 (m, 1H), 3.06 (m, 1H), 3.22 (m, 1H), 3.75 (dd, J=3.0, 9.5, 1H), 3.91 (t, J=7.0 Hz, 2H), 4.25 (br.s, 1H), 4.59 (br. s, 1H), 5.41 (m, 1H), 6.18 (d, J=15.5 Hz, 1H), 6.80 (m, 4H), 7.23 (d, J=8.5 Hz, 2H), 7.40 (d, J=8.5 Hz, 2H), 7.57 (d, J=15.5 Hz, 1H). APCI-MS (Negative): 754.3 ([M−H]⁻, 100%), 580.3 (50%); APCI-MS (Positive): 756 ([M+H]⁺, 25%), 656.4 (100%).

### Example 19

### Preparation of Compound 19

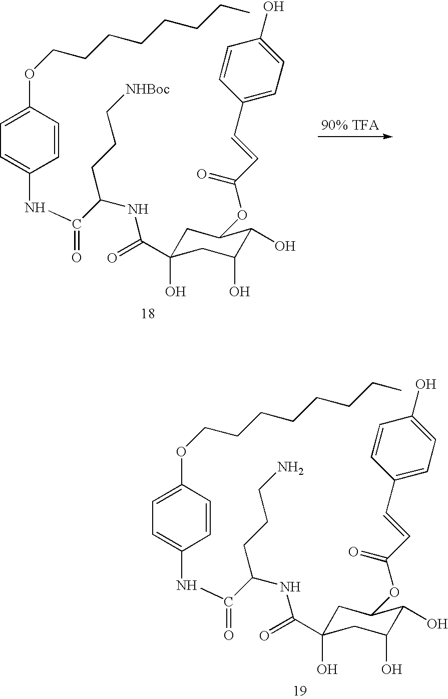

Compound 18 was treated with 90% trifluoroacetic acid under controlled conditions to obtain compound 19.

Compound 19: white solid (obtained 40 mg from 50 mg of compound 18, 92%). ¹H NMR (CDCl₃+CD₃OD, 500 MHz), δ 0.87 (t, J=7.0 Hz, 3H), 1.29 (m, 8H), 1.38 (m, 2H), 1.41 (m, 2H), 1.70 (m, 4H), 2.20 (NMR solvent signals, possibly overlapped with compound signals), 2.80 (br.s, 2H), 3.70 (br.d, J=9.0 Hz, 1H), 3.88 (t, J=7.0 Hz, 2H), 4.23 (br.s. 1H), 4.51 (br.s, 1H), 5.36 (m, 1H), 6.08 (d,J=16.0 Hz, 1H), 6.73 (m, 4H), 7.35 (d, J=8.0 Hz, 2H), 7.40 (d, J=8.0 Hz, 2H), 7.65 (d, J=16.0 Hz, 1H). ¹³C NMR (CDCl₃—CD₃OD, 125 MHz), δ 14.3, 22.9, 26.2, 29.2, 29.5, 32.0, 37.0, 38.7, 39.8, 52.9, 68.5, 70.7, 71.0, 74.0, 114.0, 114.9, 116.3, 122.0, 125.4, 129.1, 131.2, 146.0, 156.3, 159.7, 168.1, 168.5, 175.2. ESI-MS (Positive): 656.9 ([M+H]⁺, 100%). Negative HR-FAB-MS: 654.3376 ([M−H]⁻, C₃₅H₄₈O₉N₃⁻; Calc. 654.3390).

### Example 20

### Preparation of Compound 20

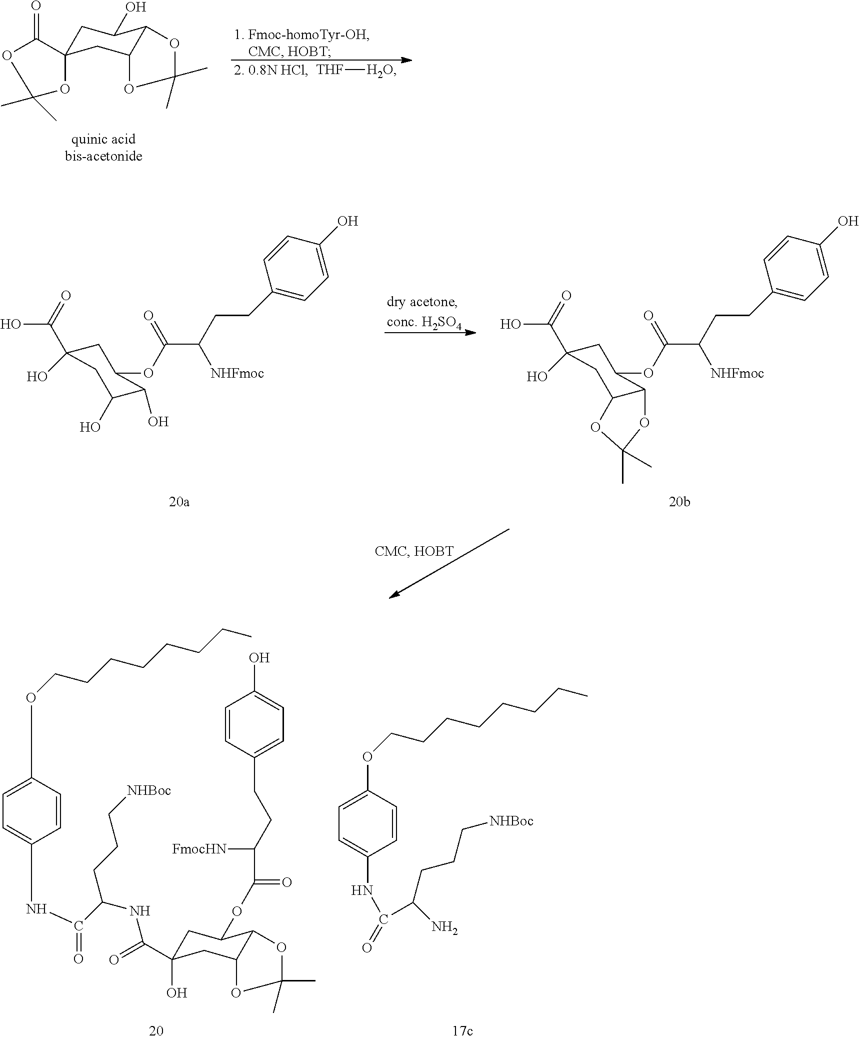

Quinic acid bis-acetonide was condensed with Fmoc-homoTyr-OH, followed by deprotection of the resulting compound with 0.8N HCl to obtain compound 20a. Compound 20a was treated with dry acetone in the presence of catalytic amount of conc. sulfuric acid to obtain acetonide 20b. Acetonide 20b was condensed with compound 17c to obtain compound 20.

Compound 20: white solid (yield 45%). ¹H NMR (CDCl₃, 500 MHz), δ 0.88 (t, J=7.0 Hz, 3H), 1.35 (m, 10H), 1.45 (m, 12H), 1.58 (m, IH), 1.59 (s, 3H), 1.78 (m, 3H), 1.95 (m, 4H), 2.19 (d, J=15.5 Hz, 1H), 2.33 (br d, J=13.5 Hz, 1H), 2.55(m, 2H), 3.01 (m, 1H), 3.20 (m, 1H), 3.41 (m, 1H), 3.93 (t, J=6.0 Hz, 2H), 4.10 (m, 1H), 4.23 (m, 1H), 4.43 (m, 2H), 4.65 (br.s, 1H), 4.82 (br.s, 1H), 5.33 (m, 1H), 5.58(m, 1H), 6.75 (d, J=8.5 Hz, 2H), 6.80 (d, J=8.5 Hz, 2H), 6.98 (d, J=8.5 Hz, 2H), 7.28 (m, 2H), 7.41 (m, 4H), 7.60 (m, 2H), 7.75 (d, J=8.5 Hz, NH), 7.78 (m, 2H), 8.62 (br.s, NH). ESI-MS (Negative): 1084.0 ([M+Cl]⁻, 100%); ESI-MS (Positive): 1049.6 ([M+H]⁺, 5%), 949.9 (100%).

### Example 21

### Preparation of Compound 21

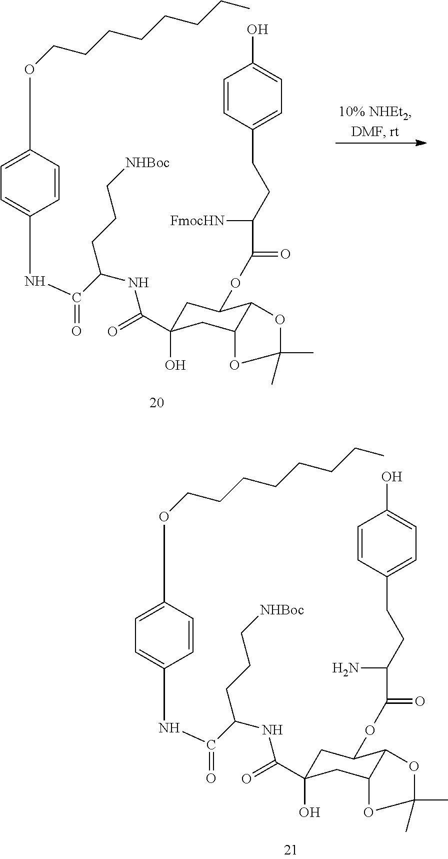

Compound 20 was treated with 10% diethylamine in DMF to obtain compound 21.

Compound 21: white solid (yield 55%). ¹H NMR (CDCl₃, 500 MHz), δ 0.88 (t, J=7.0 Hz, 3H), 1.32 (m, 11H), 1.43 (m, 10H), 1.53 (m,1H), 1.58 (s, 3H), 1.76 (m, 3H), 1.82 (m, 1H), 1.99 (m, 4H), 2.19 (d, J=15.5 Hz, 1H), 2.33 (dd, J=2.5, 13.5 Hz, 1H), 2.61(t, J=7.0Hz, 2H), 3.01 (m, 1H), 3.46 (m, 2H), 3.90 (t, J=6.0 Hz, 2H), 4.12 (m, 1H), 4.46 (m, 1H), 4.65 (br.s, 1H), 4.82 (br.s, 1H), 5.34 (m, 1H), 6.73 (d, J=8.5 Hz, 2H), 6.81 (d, J=8.5 Hz, 2H), 6.98 (d, J=8.5 Hz, 2H), 7.41 (d, J=8.5 Hz, 2H), 7.70 (d, J=8.0 Hz, NH), 8.59 (br.s, NH). ESI-MS (Negative): 862.3([M+Cl]⁻, 100%); ESI-MS (Positive): 828.0([M+H]⁺, 100%).

### Example 22

### Preparation of Compound 22

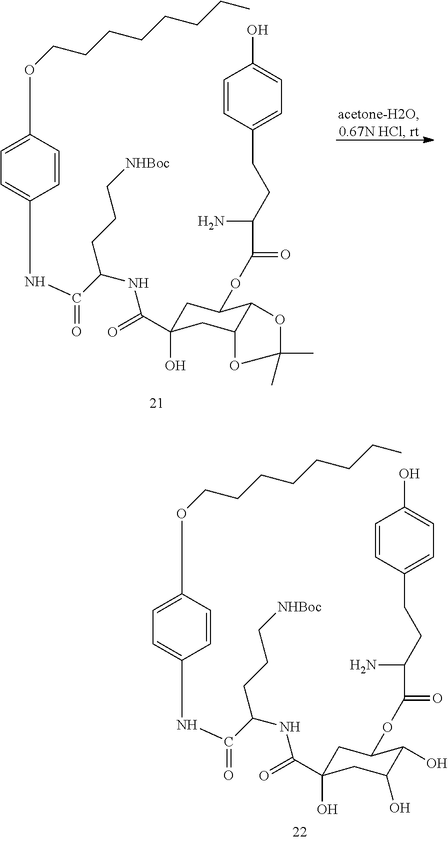

Compound 21 was treated with acetone-H₂O under acidic condition to yield compound 22.

Compound 22: white solid (yield 80%). ¹H NMR (CDCl₃+CD₃OD 500 MHz), δ 0.88 (t, J=7.0 Hz, 3H), 1.35 (m, 8H), 1.39 (m, 10H), 1.50 (m,1H), 1.65 (m, 3H), 1.88 (m, 2H), 2.01(m, 5H), 2.60(m, 2H), 3.03 (m, 1H), 3.28 (m, 1H), 3.41 (m,1H), 3.61 (m, 1H), 3.90 (t, J=6.0 Hz, 2H), 4.20 (m, 1H), 4.60 (m, 1H), 5.34 (m, 1H), 6.73 (d, J=8.5Hz, 2H), 6.81 (d, J=8.5 Hz, 2H), 6.97 (d, J=8.5 Hz, 2H), 7.41 (d, J=8.5 Hz, 2H). ESI-MS (Negative): 786.0 ([M−H]⁻, 100%); ESI-MS (Positive): 788.0 ([M+H]⁺, 100%).

### Example 23

### Preparation of Compound 23

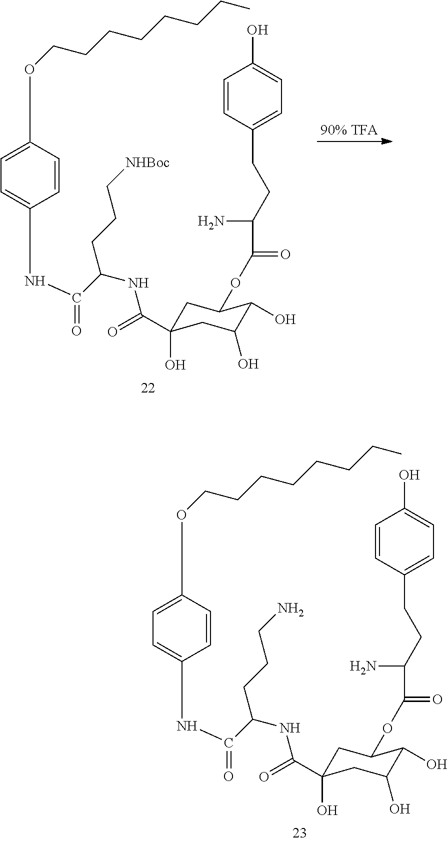

Compound 22 was treated with 90% trifluoroacetic acid under controlled conditions to obtain compound 23.

Compound 23: white solid (yield 85%). ¹H NMR (CDCl₃+CD₃OD 500 MHz), δ 0.89 (t, J=7.0 Hz, 3H), 1.31 (m, 8H), 1.45 (m, 2H), 1.76 (m,4H), 1.93 (m, 1H), 2.06 (m, 4H), 2.20 (m, 1H), 2.68 (m, 2H), 2.96 (m, 2H), 3.40 (solvent signal, possibly overlapped with signals from compound), 3.68 (m, 1H), 3.94 (H₂O signal, possibly overlapped with signals from compound), 4.27 (br s, 1H), 4.52 (m, 1H), 5.44 (m, 1H), 6.76 (d, J=8.5 Hz, 2H), 6.86 (d, J=8.5 Hz, 2H), 7.02 (d, J=8.5 Hz, 2H), 7.44 (d, J=8.5 Hz, 2H), ESI-MS (Negative): 685.7 ([M−H]⁻, 100%).

### Example 24

### In Vitro Anti-Fungal Activity of Chlorogenic Acid Derivatives

The anti-fungal activity of the compounds according to the invention were evaluated against Candida albicans (ATCC90028), Cryptococcus neoformans (ATCC32045), and Aspergillus fumigatus (ATCC13073) according to NCCLS guidelines (NCCLS reference method for broth dilution anti-fungal susceptibility testing of yeasts; approved standard-second edition, M27-A2, National Committee for Clinical Laboratory Standards, Villanova, Pa. 2003; and NCCLS reference method for broth dilution anti-fungal susceptibility testing of filamentous fungi; approved standard, M38-A, National Committee for Clinical Laboratory Standards, Villanova, Pa. 2002).

The medium required for the evaluation of anti-fungal activity was prepared as follows. RPMI 1640 broth+MOPS, pH adjusted and filter sterilized. Sabouraud Dextrose Agar (SDA) plates were used for growing fungi.

All test or candidate compounds were prepared as 5 mg/ml stock solutions in DMSO and further diluted according to the NCCLS guidelines with sterile water or appropriate diluent. A working stock of 256 μg/ml was used to prepare 1:2 serial dilutions in 96-well plates. Final MIC concentrations tested ranged from to 0.12 μg/ml to 64 μg/ml.

The inoculums of fungi were prepared by making a direct sterile water suspension of colonies/spores from SDA plates. 0.1% Tween 20 was used for A. fumigatus for better dispersal of spores in suspension. For C. albicans and C. neoformans overnight cultures were used. A. fumigatus required longer incubation. Each fungal suspension was adjusted to read between 70 and 75% transmittance at 530 nm (0.5 McFarland Standard). These suspensions were further diluted 1/1000 in appropriate broth for inoculating the 96-well plates.

All plates were incubated at 35° C. for the following times: 18 hours to 24 hours for C. albicans, 48 hours for A. fumigatus, and 48 to 72 hours for C. neoformans. The results are shown in Table 1.

TABLE 1
Anti-fungal activity of Compounds 1 to 23

| | MIC^{a} (μg/ml) | | |
|---|---|---|---|
| Compound | C. albicans | C. neoformans | A. fumigatus |
| 1 | 16 | 2 | >64 |
| 2 | 8 | 2 | >64 |
| 3 | 32 | 2 | >64 |
| 4 | 32 | 4 | >64 |
| 5 | >64 | 2 | >64 |
| 6 | >64 | 2 | >64 |
| 7 | 4 | 2 | >64 |
| 8 | 2 | 1 | >64 |
| 9 | 2 | 2 | >64 |
| 10 | 2 | 1 | >64 |
| 11 | >64 | 4 | >64 |
| 12 | 8 | 2 | >64 |
| 13 | 64 | 16 | >64 |
| 14 | 4 | 1 | 16 |
| 15 | >64 | 4 | >64 |
| 16 | 16 | 8 | 64 |
| 17 | >64 | >64 | >64 |
| 18 | >64 | >64 | >64 |
| 19 | 8 | 4 | 32 |
| 20 | >64 | >64 | >64 |
| 21 | >64 | >64 | >64 |
| 22 | >64 | 4 | >64 |
| 23 | 64 | 2 | >64 |
| Fluconazole | 0.25 | 0.5 | >64 |

^{a}MICs were read at 80% inhibition.

As can be seen from Table 1, compound 14 with a free amino group in its structure showed good activity against all the fungi tested, including A. fumigatus (MIC of 16 μg/ml), suggesting that activity against the A. fumigatus fungus may be improved by inclusion in the compound of a free amino group at R7.

With respect to activity against C. albicans, compounds 1 and 3-6comprising an acetonide group showed weaker inhibitory activity than the corresponding compounds with free hydroxyl groups (compounds 2 and 7-10), suggesting that the two hydroxyl groups in the quinic acid part of the compound improve activity against C. albicans.

### Example 25

### General Toxicity Testing of Compounds

To test the general toxicity of these compounds, Brine Shrimp Lethality assay was carried out according to Mclaughlin, J. L. in Methods in Plant Biochemistry. Ed. K. Hostettmann, Academic Press, London, 1991; Vol. 6, pp. 1-32. The compounds were tested at 100 μg/ml. The results of this assay are shown in Table 2.

TABLE 2
General Toxicity of Compounds 1 to 23

| Compound | % BL^{a} |
|---|---|
| 1 | 100.0 |
| 2 | 50.0 |
| 3 | 31.3 |
| 4 | 6.2 |
| 5 | 56.2 |
| 6 | 12.5 |
| 7 | 31.1 |
| 8 | 50.0 |
| 9 | 37.5 |
| 10 | 25.0 |
| 11 | 18.8 |
| 12 | 12.5 |
| 13 | 6.3 |
| 14 | 37.5 |
| 15 | 21.1 |
| 16 | 10.5 |
| 17 | 0.0 |
| 18 | 31.6 |
| 19 | 100 |
| 20 | 10.5 |
| 21 | 47.7 |
| 22 | 36.8 |
| 23 | 73.7 |
| Chlorogenic acid | 100 |

^{a}BL: (%) brine shrimp lethality at 100 μg/ml.

### Example 26

### Additional Testing of Anti-Fungal Activity of Compounds 8 and 12

Compounds 8 and 12 were further tested for their anti-fungal activity against two Candida species, Candida albicans and the drug resistant species Candida Krusei, in comparison with the parent compound, chlorogenic acid, and the positive control amphotericin B. The anti-fungal activity of these compounds was tested as described in Example 24. As shown in Table 3, both compounds 8 and 12 showed more potent inhibitory activity against the tested fungi but less toxicity on brine shrimp compared to chlorogenic acid as measured using the Brine Shrimp Lethality assay (see Table 2 above).

TABLE 3
Comparison of anti-fungal activity of compounds 8 and 12 with
chlorogenic acid

MIC^{a,b} (μg/ml)

| Compound | C. albicans | C. krusei |
|---|---|---|
| 8 | 4 | 2 |
| 12 | 4 | 2 |
| Chlorogenic acid | >64 | >64 |
| Amphotericin B | 0.5 | 1 |

^{a}The final concentration of anti-fungal agents was between 0.12 and 64 μg/ml. MICs were read at 80% inhibition.
^{b}Tested organisms: Candida albicans ATCC90028, Candida krusei ATCC6258.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. A compound having the structural Formula I: 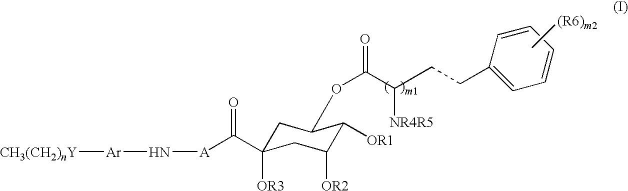
or a salt thereof, wherein:
the bond - - - is a single or a double bond;
R1, R2 and R3 are independently H, —C(O)C₁-C₆ alkyl, or C₁-C₆ alkyl; alternatively R1 and R2 are joined together to form an acetonide;
Ar is C₆-C₁₀ aryl;
Y is O, NH, S, SO, or SO₂;
n is 3 to 16;
A is either absent, or 1 to 3 amino acid residues or derivatives thereof;
R4 and R5 are independently H, C₁-C₆ alkyl or an amino protecting group;
R6 is H, —OR, —OC(O)R, —C(O)OR, —NO₂ or —NR^{a}R^{b}; wherein R is H or C₁-C₆ alkyl, R^{a} and R^{b} are independently H, C₁-C₆ alkyl or an amino protecting group;
m1 is 0 or 1; and
m2 is 1 to 5.

2. The compound according to claim 1, wherein 1 to 3 amino acid residues or derivatives thereof are each selected from threonine, a derivative of threonine, ornithine, a derivative of ornithine, lysine, a derivative of lysine, serine, a derivative of serine, aspartate, a derivative of aspartate, glutamate or a derivative of glutamate.

3. The compound according to claim 1, wherein the amino acid residue or derivative thereof is: 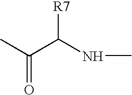
wherein R7 is —CH₂—OR, —CH(R)—OR, —CH₂—C(O)OR, —CH₂—CH₂—C(O)OR, —(CH₂)₃NR^{c}R^{d}, or —(CH₂)₄NR^{c}R^{d}, wherein R^{c} and R^{d} are independently H, C₁-C₆ alkyl, —C(O)OR or an amino protecting group and R is independently H or C₁-C₆ alkyl.

4. The compound according to claim 1, wherein said compound has the structural Formula II: 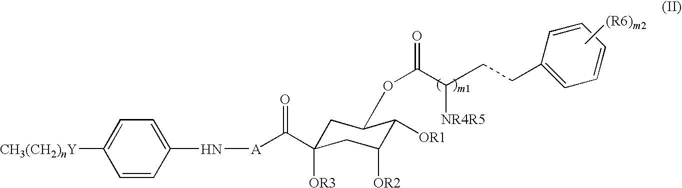
wherein the bond - - -, R1, R2, R3, R4, R5, R6, A, Y, n, m1 and m2 are as defined in claim 1.

5. The compound according to claim 1, wherein said compound has the structural Formula III: 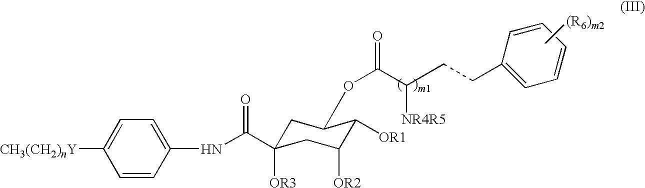
wherein the bond - - -, R1, R2 R3, R4, R5, R6, Y, n, m1 and m2 are as defined in claim 1.

6. The compound according to claim 1, wherein said compound has the structural Formula IV: 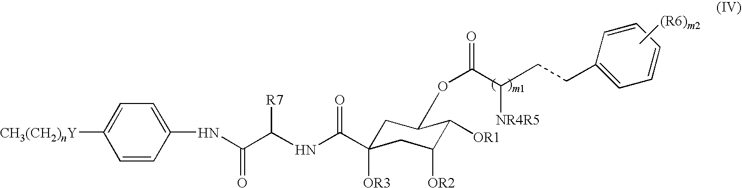
wherein the bond - - -, R1, R2, R3, R4, R5, R6, R7, Y, n, m1 and m2 are as defined in claim 1.

7. The compound according to claim 1, wherein m2 is 1 or 2.

8. The compound according to claim 7, wherein m2 is 1 and the R6 group is present at the para position of the phenyl ring.

9. The compound according to claim 7, wherein m2 is 2 and the R6 groups arc present at the 3- and 4-positions of the phenyl ring.

10. The compound according to claim 1, wherein said compound has the structural Formula IVa: 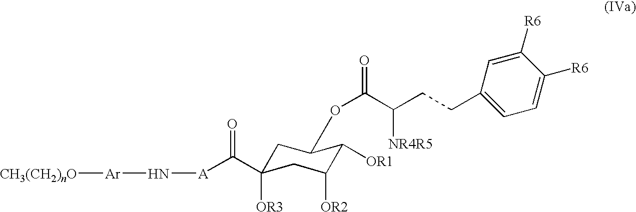
wherein the bond - - -, R1, R2, R3, R4, R5 and n are as defined in claim 1, and R6 is —OR or —OC(O)R, wherein R is H or C₁-C₆ alkyl.

11. The compound according to claim 10, wherein R6 is —OR or —OC(O)R, wherein R is H or C₁-C₆ alkyl.

12. The compound according to claim 10, wherein R6 is —OH.

13. The compound according to claim 1, wherein said compound is:

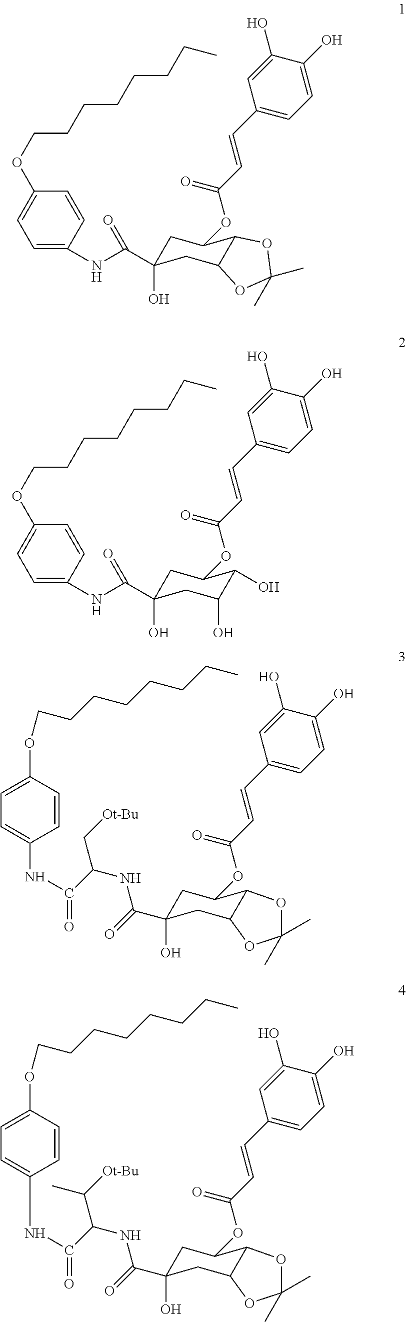

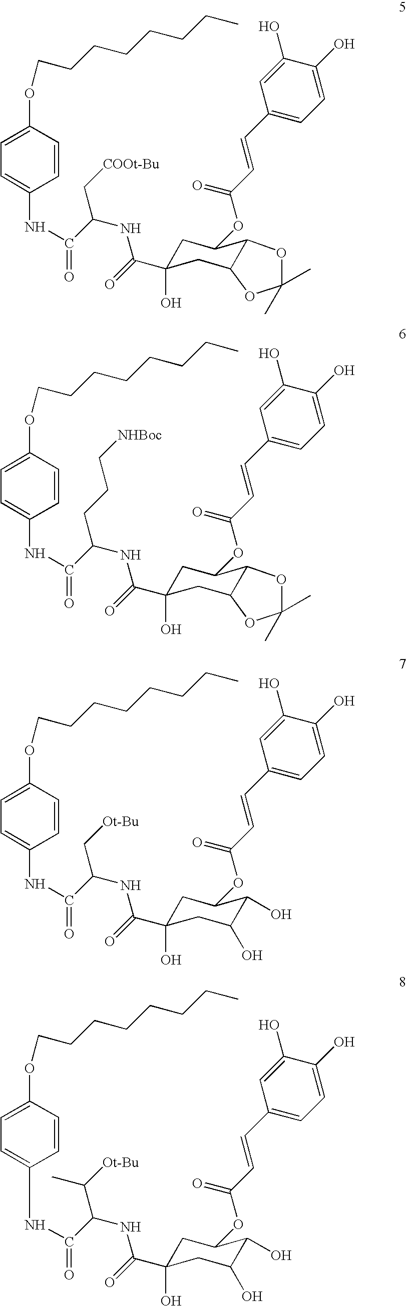

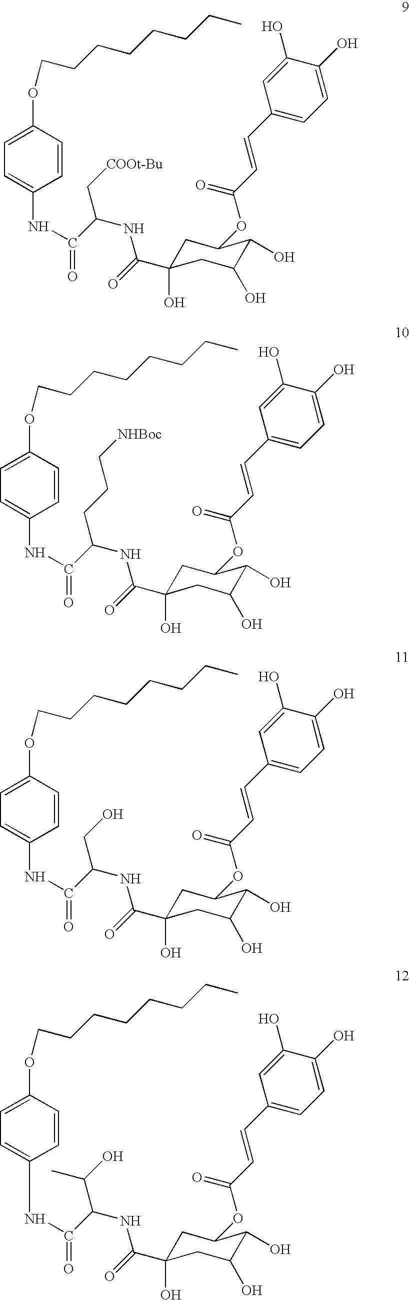

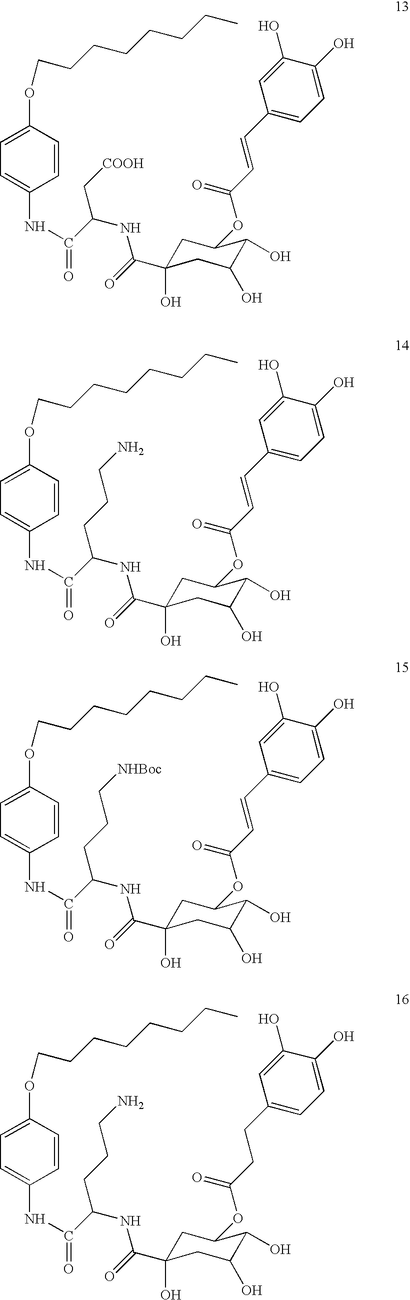

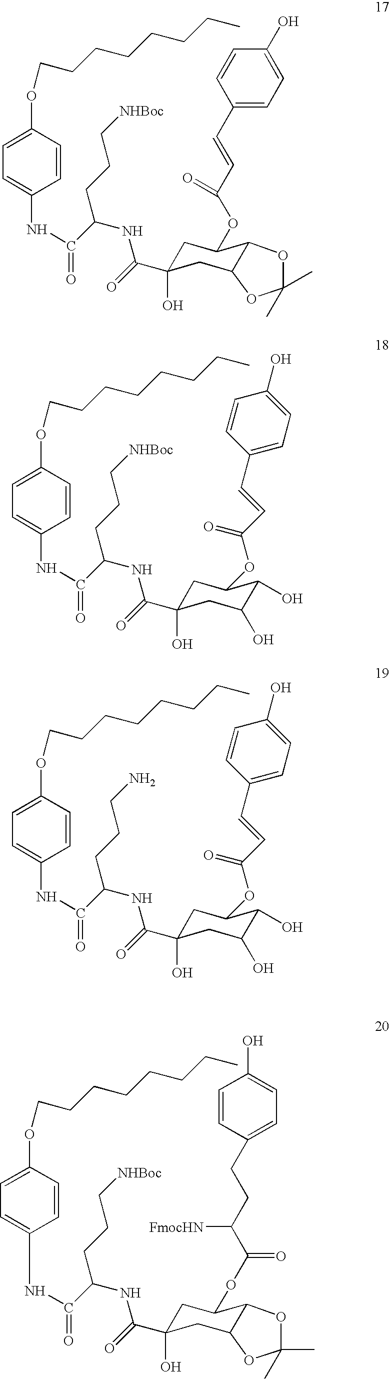

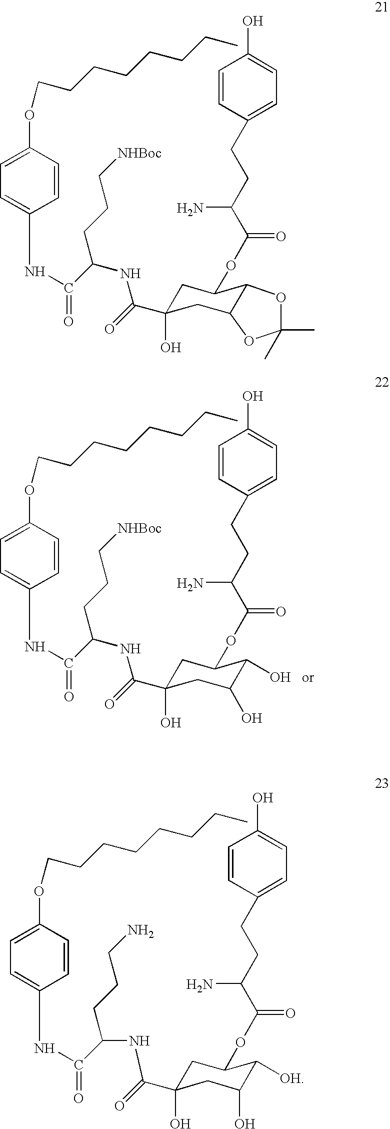

14. A composition comprising the compound according to claim 1, and a carrier.

15. The composition according to claim 14, wherein said composition is a pharmaceutical composition and said carrier is a pharmaceutically acceptable carrier.

16. The composition according to claim 14, wherein said composition is a cosmetic, personal care, household or industrial product.

17. The composition according to claim 14, wherein the composition is for inhibiting the growth of fungal cells.

18. The composition according to claim 14, further comprising one or more anti-fungal agents.

19. The compound according to claim 1 for use as an anti-fungal agent.

20-31. (canceled)

32. A method of inhibiting the growth of fungal cells comprising contacting said fungal cells with an effective amount of the compound according to claim 1.

33. The method according to claim 32, wherein said fungal cells are Candida sp., Cryptococcus sp. or Aspergillus sp. cells.

34. The method according to claim 32, wherein said fungal cells are in vivo.

35. A method of treating a fungal infection in a subject, comprising administering to the subject an effective amount of the compound according to claim 1.

36. The method according to claim 35, wherein said fungal infection is a Candida sp., Cryptococcus sp. or Aspergillus sp. infection.

37. A method of treating a fungally-related disease or disorder in a subject, comprising administering to the subject an effective amount of the compound according to claim 1.

38. The method according to claim 37, wherein said fungally-related disease or disorder is related to a Candida sp., Cryptococcus sp. or Aspergillus sp. fungus.

39. The method according to claim 32, wherein said compound is administered in combination with one or more further anti-fungal agents.

40. A kit comprising a compound according to claim 1, and optionally instructions for use.

41. The method according to claim 35, wherein said compound is administered in combination with one or more further anti-fungal agents.

42. The method according to claim 37, wherein said compound is administered in combination with one or more further anti-fungal agents.
